(19) United States
(12) Patent Application Publication
(10) Pub. No.: US 2008/0318985 A1
(43) Pub. Date: Dec. 25, 2008

(54) Novel 2-Aminopyrimidinone Or 2-Aminopyridinone Derivatives and Their Use

(75) Inventors: Jeffrey Albert, Wilmington, DE (US); Phil Edwards, Wilmington, DE (US); James Empfield, Wilmington, DE (US)

Correspondence Address: PEPPER HAMILTON LLP, 400 BERWYN PARK, 899 CASSATT ROAD, BERWYN, PA, 19312-1183, US

(73) Assignees: AstraZeneca AB, Sodertalje (SE); Astex Therapeutics Limited, Cambridge (GB)

(21) Appl. No.: 12/093,634

(22) PCT Filed: Nov. 13, 2006
(86) PCT No.: PCT/SE2006/001282

§ 371 Date: Jun. 3, 2008

Related U.S. Application Data
(60) Provisional application No. 60/737,399, filed on Nov. 15, 2005.

(51) Int. Cl. A61K 31/513; C07D 239/47

(52) U.S. Cl. 514/272; 544/321

(57) ABSTRACT

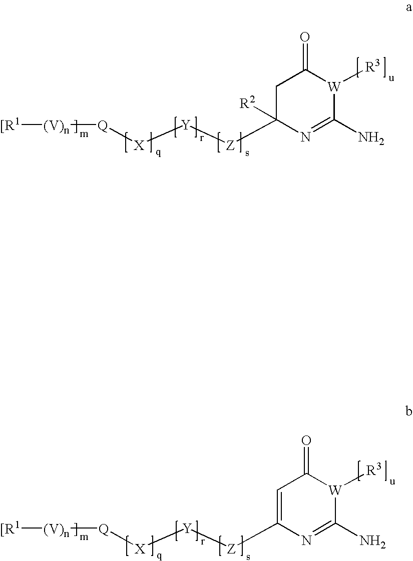

This invention relates to novel compounds having the structural formula Ia or Ib below: (Ia); (Ib) and to their pharmaceutically acceptable salts, compositions and methods of use. These novel compounds provide a treatment or prophylaxis of cognitive impairment, Alzheimer Disease, neurodegeneration and dementia.

The present invention relates to novel compounds, their pharmaceutical compositions. In addition, the present invention relates to therapeutic methods for the treatment and/or prevention of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

### BACKGROUND OF THE INVENTION

Several groups have identified and isolated aspartate proteinases that have β-secretase activity (Hussain et al., 1999; Lin et. al, 2000; Yan et. al, 1999; Sinha et. al., 1999 and Vassar et. al., 1999). β-secretase is also known in the literature as Asp2 (Yan et. al, 1999), Beta site APP Cleaving Enzyme (BACE) (Vassar et. al., 1999) or memapsin-2 (Lin et al., 2000). BACE was identified using a number of experimental approaches such as EST database analysis (Hussain et al. 1999); expression cloning (Vassar et al. 1999); identification of human homologs from public databases of predicted C. elegans proteins (Yan et al. 1999) and finally utilizing an inhibitor to purify the protein from human brain (Sinha et al. 1999). Thus, five groups employing three different experimental approaches led to the identification of the same enzyme, making a strong case that BACE is a β-secretase. Mention is also made of the patent literature: WO96/40885, EP871720, U.S. Pat. Nos. 5,942,400 and 5,744,346, EP855444, U.S. Pat. No. 6,319,689, WO99/64587, WO99/31236, EP1037977, WO00/17369, WO01/23533, WO0047618, WO00/58479, WO00/69262, WO01/00663, WO01/00665, U.S. Pat. No. 6,313,268.

BACE was found to be a pepsin-like aspartic proteinase, the mature enzyme consisting of the N-terminal catalytic domain, a transmembrane domain, and a small cytoplasmic domain. BACE has an optimum activity at pH 4.0-5.0 (Vassar et al, 1999)) and is inhibited weakly by standard pepsin inhibitors such as pepstatin. It has been shown that the catalytic domain minus the transmembrane and cytoplasmic domain has activity against substrate peptides (Lin et al, 2000). BACE is a membrane bound type 1 protein that is synthesized as a partially active proenzyme, and is abundantly expressed in brain tissue. It is thought to represent the major β-secretase activity, and is considered to be the rate-limiting step in the production of amyloid-β-protein (Aβ). It is thus of special interest in the pathology of Alzheimer's disease, and in the development of drugs as a treatment for Alzheimer's disease.

Aβ or amyloid-β-protein is the major constituent of the brain plaques which are characteristic of Alzheimer's disease (De Strooper et al, 1999). Aβ is a 39-42 residue peptide formed by the specific cleavage of a class I transmembrane protein called APP, or amyloid precursor protein. Aβ-secretase activity cleaves this protein between residues Met671 and Asp672 (numbering of 770aa isoform of APP) to form the N-terminus of Aβ. A second cleavage of the peptide is associated with γ-secretase to form the C-terminus of the Aβ peptide.

Alzheimer's disease (AD) is estimated to afflict more than 20 million people worldwide and is believed to be the most common form of dementia. Alzheimer's disease is a progressive dementia in which massive deposits of aggregated protein breakdown products—amyloid plaques and neurofibrillary tangles accumulate in the brain. The amyloid plaques are thought to be responsible for the mental decline seen in Alzheimer's patients.

The likelihood of developing Alzheimer's disease increases with age, and as the aging population of the developed world increases, this disease becomes a greater and greater problem. In addition to this, there is a familial link to Alzheimer's disease and consequently any individuals possessing the double mutation of APP known as the Swedish mutation (in which the mutated APP forms a considerably improved substrate for BACE) have a much greater chance of developing AD, and also of developing it at an early age (see also U.S. Pat. No. 6,245,964 and U.S. Pat. No. 5,877,399 pertaining to transgenic rodents comprising APP-Swedish). Consequently, there is also a strong need for developing a compound that can be used in a prophylactic fashion for these individuals.

The gene encoding APP is found on chromosome 21, which is also the chromosome found as an extra copy in Down's syndrome. Down's syndrome patients tend to acquire Alzheimer's disease at an early age, with almost all those over 40 years of age showing Alzheimer's-type pathology (Oyama et al., 1994). This is thought to be due to the extra copy of the APP gene found in these patients, which leads to overexpression of APP and therefore to increased levels of APPβ causing the high prevalence of Alzheimer's disease seen in this population. Thus, inhibitors of BACE could be useful in reducing Alzheimer's-type pathology in Down's syndrome patients.

Drugs that reduce or block BACE activity should therefore reduce Aβ levels and levels of fragments of Aβ in the brain, or elsewhere where Aβ or fragments thereof deposit, and thus slow the formation of amyloid plaques and the progression of AD or other maladies involving deposition of Aβ or fragments thereof (Yankner, 1996; De Strooper and Konig, 1999). BACE is therefore an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

It would therefore be useful to inhibit the deposition of Aβ and portions thereof by inhibiting BACE through inhibitors such as the compounds provided herein.

The therapeutic potential of inhibiting the deposition of Aβ has motivated many groups to isolate and characterize secretase enzymes and to identify their potential inhibitors (see, e.g., WO01/23533 A2, EP0855444, WO00/17369, WO00/58479, WO00/47618, WO00/77030, WO01/00665, WO01/00663, WO01/29563, WO02/25276, U.S. Pat. No. 5,942,400, U.S. Pat. No. 6,245,884, U.S. Pat. No. 6,221,667, U.S. Pat. No. 6,211,235, WO02/02505, WO02/02506, WO02/02512, WO02/02518, WO02/02520, WO02/14264, WO05/058311, WO 05/097767, US2005/0282826).

The compounds of the present invention show improved properties compared to the potential inhibitors known in the art, e.g. improved hERG selectivity.

### DISCLOSURE OF THE INVENTION

Provided herein are novel compounds of structural formula Ia or Ib:

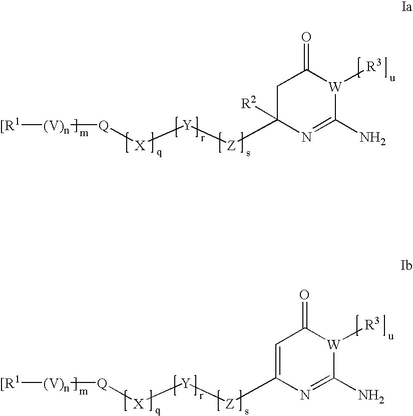

or a pharmaceutically acceptable salt, tautomer or in vivo-hydrolysable precursor thereof, wherein:

### W is C or N;

Q is selected from C₃₋₁₂cycloalkyl, C₃₋₁₂cycloalkenyl, C₆₋₁₄aryl, or C₅₋₁₅heterocyclyl;
each R¹ is, independently, selected from H, halogen, C₂₋₆alkenyl, C₁₋₆alkyl, C₃₋₁₂cycloalkyl, C₆₋₁₀aryl, C₁₋₆alkyl-C₆₋₁₀aryl, or C₅₋₁₅heterocyclyl wherein the C₁₋₆alkyl, the C₃₋₁₂cycloalkyl, the C₆₋₁₀aryl, the C₁₋₆alkyl-C₆₋₁₀aryl, or the C₅₋₁₅heterocyclyl is optionally substituted by 1, 2, or 3 substitutents independently selected from: halogen, CN, NH₂, OH, COOH, OC₁₋₆alkyl, CH₂OH, SO₂H, S(═O), C₂₋₆alkenyl, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂, S(═O)C₁₋₆alkyl-R^{a}, S(═O)NHC₁₋₆alkyl-R^{a}, S(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-R^{a}, N(C₁₋₆alkyl-R^{a})₂, NHC(═O)C₁₋₆alkyl, C₆— laryl-R^{a}, OC₆₋₁₀aryl-R^{a}, C(═O)C₆₋₁₀aryl-R^{a}, C(═O)OC₆₋₁₀aryl-R^{a}, C(═O)NHC₆₋₁₆aryl-R^{a}, C(═O)N(C₆₋₁₀aryl-R^{a})₂, S(═O)C₆₋₁₀aryl-R^{a}, S(═O)NHC₆₋₁₀aryl-R^{a}, S(═O)N(C₆₋₁₀aryl-R^{a})₂, SO₂C₆₋₁₀aryl-R^{a}, SO₂NHC₆₋₁₀aryl-R^{a}, SO₂N(C₆₋₁₀aryl-R^{a})₂, NH(C₆₋₁₀aryl)-R^{a}, N(C₆₋₁₀aryl-R^{a})₂, NC(═O)C₆₋₁₀aryl, C₅₋₆heterocyclyl-R^{a}, OC₅₋₆heterocyclyl-W, C(═O)C₅₋₆heterocyclyl-R^{a}, C(═O)OC₅₋₆heterocyclyl-IV, C(═O)NHC₅₋₆heterocyclyl-R^{a}, C(═O)N(C₅₋₆heterocyclyl-R^{a})₂, S(═O)NHC₅₋₆heterocyclyl-, S(═O)N(C₅₋₆heterocyclyl-R^{a})₂, SO₂C₅₋₆heterocyclyl-IV, SO₂NHC₅₋₆heterocyclyl-IV, SO₂N(C₅₋₆heterocyclyl-R^{a})₂, NH(C₅₋₆heterocyclyl-R^{a}); N(C₅₋₆heterocyclyl-R^{a})₂, NHC(═O)C₅₋₆heterocyclyl, SO₂R^{a}, S(═O)R^{a}, N(C₁₋₆alkyl-IV)(C₆₋₁₀aryl-IV), N(C₁₋₆alkyl-R^{a})(C₆₋₁₀heteroaryl-R^{a}), N(C₆₋₁₀aryl-R^{a})(C₆₋₁₀heteroaryl-R^{a}), C(═O)(C₁₋₆alkyl-R^{a})(C₆₋₁₀aryl-R^{a}), C(═O)(C₁₋₆alkyl-R^{a})(C₆₋₁₀heteroaryl-R^{a}), C(═O)(C₆₋₁₀aryl-R^{a})(C₆₋₁₀heteroaryl-R^{a}), C(═O)O(C₁₋₆alkyl-R^{a})(C₆₋₁₀aryl-R^{a}), C(═O)O(C₁₋₆alkyl-R^{a})(C₆₋₁₀heteroaryl-R^{a}), C(═O)O(C₆₋₁₀aryl-R^{a})(C₆₋₁₀heteroaryl-R^{a}), S(═O)(C₁₋₆alkyl-R^{a})(C₆₋₁₀aryl-R^{a}), S(═O)(C₁₋₁₀alkyl-R^{a})(C₆₋₁₀heteroaryl-R^{a}), S(═O)(C₆₋₁₀aryl-R^{a})(C₆₋₁₀heteroaryl-R^{a}), SO₂(C₁₋₆alkyl-R^{a})(C₆₋₁₀aryl-R^{a}), SO₂(C₁₋₆alkyl-R^{a})(C₆₋₁₀heteroaryl-R^{a}), or SO₂(C₆₋₁₀aryl-R^{a})(C₆₋₁₀heteroaryl-R^{a});
each R^{a} is, independently, selected from H, halogen, CN, NH₂, OH, C₁₋₆alkyl, OC₁₋₆alkyl, C(═O)C₁₋₆alkyl, C(═O)OC₁₋₆alkyl, C(═O)NH₂, C(═O)NHC₁₋₆alkyl, C(═O)N(C₁₋₆alkyl)₂, SOC₁₋₆alkyl, SONHC₁₋₆alkyl, SON(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NC(═O)C₁₋₆alkyl, C₅₋₆aryl, OC₅₋₆aryl, C(═O)C₅₋₆aryl, C(═O)OC₅₋₆aryl, C(═O)NH₂, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NC(═O)C₅₋₆aryl, C₅₋₆heterocyclyl, OC₅₋₆heterocyclyl, C(═O)C₅₋₆heterocyclyl, C(═O)OC₅₋₆heterocyclyl, C(═O)NH₂, C(═O)NHC₅₋₆heterocyclyl, C(═O)N(C₅₋₆heterocyclyl)₂, S(═O)C₅₋₆heterocyclyl, S(═O)NHC₅₋₆heterocyclyl, S(═O)N(C₅₋₆heterocyclyl)₂, SO₂NHC₅₋₆heterocyclyl, SO₂N(C₅₋₆heterocyclyl)₂, NH(C₅₋₆heterocyclyl), N(C₅₋₆heterocyclyl)₂, NC(═O)C₅₋₆heterocyclyl, C(═O)NHC₁₋₆alkylC₅₋₆aryl, NR^{b}R^{b}, C(═O)R^{b}, C(═O)NR^{b}R^{b}, OC(═O)NR^{b}R^{b}, S(═O)R^{b}, S(═O)NR^{b}R^{b}, or SO₂NR^{b}R^{b};
each R^{b} is, independently, selected from H, C₁₋₆alkyl, C₅₋₆aryl, or C₅₋₆heterocyclyl;
each V is, independently, selected from NH, O, S, S(═O), SO₂, NHS(═O), NHSO₂, S(═O)NH, SO₂NH, NHC(—O), C(═O)NH, NR^{a}SO₂, NR^{a}S(═O), NR^{a}C(O), C(O)NR^{a}, S(O)₂NR^{a}, S(═O)NR^{a}, OC₁₋₆alkylenyl, C₂₋₆alkenylenyl or C₁₋₆alkylenyl, wherein the OC₁₋₆alkylenyl, C₂₋₆alkenylenyl, and C₁₋₆alkylenyl is optionally substituted by 1, 2, or 3 substitutents independently selected from R^{a};
X and Y are each independently selected from NH, O, S, S(═O), SO₂, NHS(═O), NHSO₂, S(═O)NH, SO₂NH, NHC(═O), C(═O)NH, NR^{a}SO₂, NR^{a}S(═O), NR^{a}C(O), C(O)NR^{a}, S(O)₂NR^{a}, S(═O)NR^{a}, or C₁₋₆alkyl wherein the C₁₋₆alkyl is optionally substituted by 1, 2, or 3 substituents independently selected from R^{a};

### Z is;

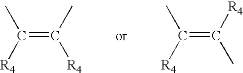

m is 0, 1, 2 or 3;
n, q, r, and u are each, independently, 0 or 1;
s is 1 or 2;
R² is selected from H, halogen, C₁₋₆alkyl, C₃₋₁₂cycloalkyl, C₆₋₁₀aryl, C₁₋₆alkyl-C₆₋₁₀aryl, C₅₋₁₀heterocyclyl, or C₁₋₆alkyl-C₅₋₁₀heterocyclyl wherein the C₁₋₆alkyl, C₃₋₁₂cycloalkyl, C₆₋₁₀aryl, C₁₋₆alkyl-C₆₋₁₀aryl, C₅₋₁₀heterocyclyl, and C₁₋₆alkyl-C₅₋₁₀heterocyclyl is optionally substituted by 1, 2, or 3 substitutents independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂ S(═O)C₁₋₆alkyl-R^{a}, S(═O)NHC₁₋₆alkyl-R^{a}, S(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-R^{a}, N(C₁₋₆alkyl-R^{a})₂, NHC(═O)C₁₋₆alkyl, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl-R^{a})₂, S(═O)C₅₋₆aryl-R^{a}, S(═O)NHC₅₋₆aryl-R^{a}, S(═O)N(C₅₋₆aryl-R^{a})₂, SO₂C₅₋₆aryl-R^{a}, SO₂NHC₅₋₆aryl-R^{a}, SO₂N(C₅₋₆aryl-R^{a})₂, NH(C₅₋₆aryl)-R^{a}, N(C₅₋₆aryl-R^{a})₂, NHC(═O)C₅₋₆aryl, C₅₋₆heterocyclyl-R^{a}, OC₅₋₆heterocyclyl-IV, C(═O)C₅₋₆heterocyclyl-IV, C(═O)OC₅₋₆heterocyclyl-IV, C(═O)NH₂, C(═O)NHC₅₋₆heterocyclyl-R^{a}, C(═O)N(C₅₋₆heterocyclyl-R^{a})₂, SO₂C₅₋₆heterocyclyl-IV, SO₂NHC₅₋₆heterocyclyl-R^{a}), SO₂N(C₅₋₆heterocyclyl-R^{a})₂ S(═O)C₅₋₆heterocyclyl-R^{a}, S(═O)N(C₅₋₆heterocyclyl-R^{a})₂, NH(C N(C₆heterocyclyl-R^{a})₂, or NHC(═O)C₅₋₆heterocyclyl;
R³ is selected from R¹, C₁₋₆alkylR^{c}, C₁₋₆alkylNR^{c}R^{c}, C₁₋₆alkylOR^{c}, C₁₋₆alkylSR^{c}, C₁₋₆alkylNHC₁₋₆alkylC₅₋₆arylR^{d}, C₁₋₆alkylNHC₆₋₁₀arylR^{d}, C₁₋₆alkylNHC(O)C₆₋₁₀arylR^{d}, C₁₋₆alkylOC₁₋₆alkylC₅₋₆arylR^{d}, C₁₋₆alkylSC₁₋₆alkylC₅₋₆arylR^{d}, C₁₋₆alkylC₅₋₉heterocyclylR^{d}, C₁₋₆alkylC₃₋₉cycloalkylR^{d}, C₁₋₆alkylNHC₁₋₆alkylC₅₋₆heterocyclylR^{d}, C₁₋₆alkylNHC₅₋₉heterocyclyl(R^{d})ₜ, C₁₋₆alkylNHC(O)C₅₋₉heterocyclylR^{d}, C₁₋₆alkylOC₁₋₆alkylC₅₋₉heterocyclylR^{d}, C₁₋₆alkylSC₁₋₆alkylC₅₋₉heterocyclylR^{d}, C₁₋₆alkylNHC₁₋₆alkylC₃₋₉cycloalkylR^{d}, C₁₋₆alkylOC₁₋₆alkylC₃₋₉cycloalkylR^{d}, or C₁₋₆alkylSC₁₋₆alkylC₃₋₉cycloalkylR^{d};
each R⁴ is, independently, selected from H, halogen, C₁₋₆alkyl, C₃₋₁₂cycloalkyl, C₆₋₁₀aryl, C₁₋₆alkyl-C₆₋₁₀aryl, C₅₋₁₀heterocyclyl, or C₁₋₆alkyl-C₅₋₁₀heterocyclyl wherein the C₁₋₆alkyl, C₃₋₁₂cycloalkyl, C₆₋₁₀aryl, C₁₋₆alkyl-C₆₋₁₀aryl, C₅₋₁₀heterocyclyl, and C₁₋₆alkyl-C₅₋₁₀heterocyclyl is optionally substituted by 1, 2, or 3 substitutents independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(—O)NH₂, C(═O)NHC₁₋₆alkyl-R^{a}, C(═O)N(C₁₋₆alkyl-R^{a})₂ S(═O)C₁₋₆alkyl-R^{a}, S(═O)NHC₁₋₆alkyl-R^{a}, S(═O)N(C₁₋₆alkyl-R^{a})₂, SO₂C₁₋₆alkyl-R^{a}, SO₂NHC₁₋₆alkyl SO₂N(C₁₋₆alkyl-R^{a})₂, NH(C₁₋₆alkyl)-R^{a}, N(C₁₋₆alkyl-R^{a})₂, or NHC(═O)C₁₋₆alkyl;
t is 0, 1, 2, 3, 4 or 5;
each R^{c} is, independently, selected from H, C(═O)C₁₋₄alkyl, C(═O)C₁₋₄alkylOC₁₋₄alkyl, C(═O)C₁₋₄alkylC(═O)OC₁₋₄alkyl, C(═O)C₁₋₄alkylC(═O)OH, C(═O)C₁₋₄alkylOC(═O)C₁₋₄alkyl, C₅₋₆arylR^{d}, C₅₋₉heterocyclylR^{d}, C₃₋₉cycloalkylR^{d}, C(═O)C₅₋₆arylR^{d}, C(═O)C₅₋₉heterocyclylR^{d}, C(═O)C₃₋₉cycloalkylR^{d}, C₁₋₄alkyl-C₅₋₆arylR^{d}, C₁₋₄alkyl-C₅₋₉heterocyclylR^{d}, or C₁₋₄alkyl-C₃₋₉cycloalkylR¹; and
R^{d} is selected from H, C₁₋₃alkyl, NH₂, OH, COOH, OC₁₋₃alkyl, or OC₁₋₃alkylOH;
provided that when the compound has formula Ib, W is N, u is 1, and R³ is H, then [R¹-(V)ₙ]ₘ-Q is other than polyC₁₋₄alkyl substituted cyclohexenyl or a nitro substituted furyl.

In some embodiments, the compound has the structure of formula Ia.

In some embodiments, the compound has the structure of formula Ib.

In some embodiments, W is N.

In some embodiments, R³ is selected from H, C₁₋₆alkyl, C₁₋₆alkylNR^{c}R^{c}, C₁₋₆alkylOR^{c}, C₁₋₆alkylNHC₁₋₆alkylC₆₋₁₀arylR^{d}, C₁₋₆alkylNHC(O)C₆₋₁₀arylR^{d}, C₁₋₆alkylOC₁₋₆alkylC₅₋₆arylR^{d}, C₁₋₆alkylC₆₋₁₀arylR^{d}, C₁₋₆alkylC₅₋₉heterocyclylR^{d}, or C₁₋₆alkylC₃₋₉cycloalkylR^{d}. In some embodiments, R³ is selected from H, C₁₋₆alkyl, C₁₋₆alkylNR^{c}R^{c}, or C₁₋₆alkyl-C₅₋₉heterocyclyl. In some embodiments, R³ is C₁₋₃alkyl.

In some embodiments, Q is C₆₋₁₀aryl, C₃₋₁₀cycloalkyl or C₃₋₁₀cycloalkenyl. In some embodiments, Q is C₆aryl or C₃₋₁₀cycloalkenyl.

In some embodiments, -[X]_{q}-[Y] is OC₁₋₃alkyl, N(C₁₋₃alkyl)C₁₋₃alkyl, C₁₋₃alkylOC₁₋₃alkyl, C₁₋₃alkylN(H)C₁₋₃alkyl or C₁₋₃alkyl optionally substituted by OH.

In some embodiments, q is 0, r is 0, and each R⁴ is H.

In some embodiments, m is 1, V is S, n is 0 or 1, and R¹ is C₆₋₁₀aryl or C₅₋₁₅heterocyclyl, wherein each the aryl and heterocyclyl is optionally substituted by 1 or 2 substituents independently selected from: halogen, CN, C₁₋₄alkyl, C₁₋₄haloalkyl, OC₁₋₄alkyl, OC₁₋₄haloalkyl, —C(O)H, COOH, OC₁₋₄alkyl-C₆₋₁₀aryl, OH, NHC(═O)C₁₋₄alkyl and —C₆aryl-OC₁₋₄alkyl.

In some embodiments, m is 1, n is 0, and R¹ is C₆₋₁₀aryl, wherein the aryl is optionally substituted by 1 or 2 substituents independently selected from: halogen, CN, C₁₋₄alkyl, C₁₋₄haloalkyl, OC₁₋₄alkyl, OC₁₋₄haloalkyl, —C(O)H, COOH, OC₁₋₄alkyl-C₆₋₁₀aryl, OH, NHC(═O)C₁₋₄alkyl and —C₆aryl-OC₁₋₄alkyl.

In some embodiments, R¹ is, independently, selected from H, halogen, C₆aryl, or C₅₋₆heterocyclyl wherein the C₆aryl, or C₅₋₆heterocyclyl is optionally substituted by 1, 2, or 3 substituents, independently, selected from: halogen, OH, NH₂, CN, C(═O)NH₂, C₁₋₆alkyl, OC₁₋₆alkyl, C₁₋₄alkylOH, C₁₋₄alkylOC₁₋₃alkyl, CH₂OH, SO₂H, SO₂NHC(CH₃)₃, SO₂C₁₋₆alkyl, SO₂NHC₁₋₆alkyl, OC₁₋₃alkylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₅₋₆heterocyclyl, OC₅₋₆aryl, —C₆aryl-OC₁₋₄alkyl or OC₁₋₆alkyl-C₅₋₆aryl; R² is H or C₁₋₆alkyl; R³ is H or C₁₋₃alkyl; and each R⁴ is H.

In some embodiments, Q is C₆aryl or C₅₋₉heterocyclyl; W is N; R¹ is, independently, selected from H, halogen, C₆aryl, or C₅₋₆heterocyclyl wherein the C₆aryl, or C₅₋₆heterocyclyl is optionally substituted by 1, 2, or 3 substituents, independently, selected from: halogen, OH, NH₂, CN, C(═O)NH₂, C₁₋₆alkyl, OC₁₋₆alkyl, C₁₋₄alkylOH, C₁₋₄alkylOC₁₋₃alkyl, CH₂OH, SO₂H, SO₂NHC(CH₃)₃, SO₂C₁₋₆alkyl, SO₂NHC₁₋₆alkyl, OC₁₋₃alkylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₅₋₆heterocyclyl, OC₅₋₆aryl, —C₆aryl-OC₁₋₄alkyl or OC₁₋₆alkyl-C₅₋₆aryl; and R² is C₁₋₃alkyl.

In some embodiments, Q is C₆₋₁₀aryl; W is N; —[X]_{q}-[Y]ᵣ is OC₁₋₃alkyl; m is 1; n is 0; and R¹ is C₆₋₁₀aryl optionally substituted by 1 or 2 substituents independently selected from: OC₁₋₄alkyl and —C₆aryl-OC₁₋₄alkyl.

In some embodiments, Q is C₃₋₁₀cycloalkenyl; W is N; -[X]_{q}-[Y]ᵣ- is absent; m is 1; n is 0; and R¹ is C₆₋₁₀aryl optionally substituted by 1 or 2 substituents independently selected from: OC₁₋₄alkyl and —C₆aryl-OC₁₋₄alkyl.

In some embodiments, Q is C₆₋₁₀aryl, C₃₋₁₀cycloalkyl or C₃₋₁₀cycloalkenyl; W is N; —[X]_{q}-[Y]ᵣ- is OC₁₋₃alkyl, N(C₁₋₃alkyl)C₁₋₃alkyl, C₁₋₃alkylOC₁₋₃alkyl, C₁₋₃alkylN(H)C₁₋₃alkyl or C₁₋₃alkyl optionally substituted by OH; m is 1; V is S; n is 0 or 1; and R¹ is C₆₋₁₀aryl or C₅₋₁₅heterocyclyl, wherein each the aryl and heterocyclyl is optionally substituted by 1 or 2 substituents independently selected from: halogen, CN, C₁₋₄alkyl, C₁₋₄haloalkyl, OC₁₋₄alkyl, OC₁₋₄haloalkyl, —C(O)H, COOH, OC₁₋₄alkyl-C₆₋₁₀aryl, OH, NHC(═O)C₁₋₄alkyl and —C₆aryl-OC₁₋₄alkyl.

In some embodiments, the compound is
2-Amino-6-[(Z)-2-(3′-methoxybiphenyl-3-yl)vinyl]-3-methylpyrimidin-4(3H)-one;
2-Amino-6-[(E)-2-(3′-methoxybiphenyl-3-yl)vinyl]-3-methylpyrimidin-4(3H)-one;
2-Amino-6-[(Z)-2-(3-bromophenyl)vinyl]-3-methylpyrimidin-4(3H)-one and
2-Amino-6-[(E)-2-(3-bromophenyl)vinyl]-3-methylpyrimidin-4(3H)-one
   or a pharmaceutically acceptable salt, tautomer, or in vivo-hydrolysable precursor thereof.

The present invention further provides compositions comprising a compound of formula Ia or Ib, or a pharmaceutically acceptable salt, tautomer or in vivo-hydrolysable precursor thereof, and at least one pharmaceutically acceptable carrier, diluent or excipient.

The present invention further provides methods of modulating activity of BACE comprising contacting the BACE with a compound of formula Ia or Ib, or a pharmaceutically acceptable salt, tautomer or in vivo-hydrolysable precursor thereof.

The present invention further provides methods of treating or preventing an Aβ-related pathology in a patient, comprising administering to the patient a therapeutically effective amount of a compound of formula Ia or Ib, or a pharmaceutically acceptable salt, tautomer or in vivo-hydrolysable precursor thereof.

The present invention further provides a compound of formula Ia or Ib, or a pharmaceutically acceptable salt, tautomer or in vivo-hydrolysable precursor thereof, described herein for use as a medicament.

The present invention further provides a compound of formula Ia or Ib, or a pharmaceutically acceptable salt, tautomer or in vivo-hydrolysable precursor thereof, described herein for the manufacture of a medicament.

### DETAILED DESCRIPTION OF THE INVENTION

Provided herein are novel compounds of structural formula Ia or Ib:

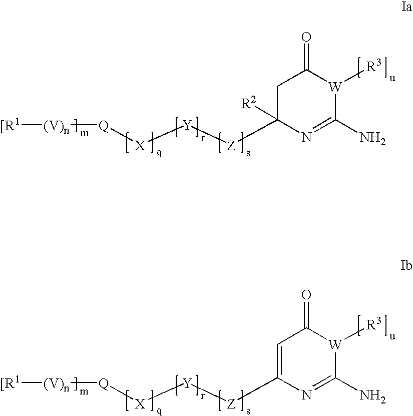

or a pharmaceutically acceptable salt, tautomer or in vivo-hydrolysable precursor thereof.

In some embodiments, W is C or N. In some embodiments, W is N.

In some embodiments, Q is selected from C₃₋₁₂cycloalkyl, C₃₋₁₂cycloalkenyl, C₆₋₁₄aryl, or C₅₋₁₅heterocyclyl, or any subgroup thereof. In some embodiments, Q is C₆₋₁₀aryl, C₃₋₁₀cycloalkyl or C₃₋₁₀cycloalkenyl. In some embodiments, Q is C₆aryl or C₃₋₁₀cycloalkenyl. In some embodiments, Q is C₆aryl or C₅₋₉heterocyclyl. In some embodiments, Q is C₆₋₁₀aryl. In some embodiments, Q is C₃₋₁₀cycloalkenyl.

In some embodiments, each R¹ is, independently, selected from H, halogen, C₂₋₆alkenyl, C₁₋₆alkyl, C₃₋₁₂cycloalkyl, C₆₋₁₀aryl, C₁₋₆alkyl-C₆₋₁₀aryl, or C₅₋₁₅heterocyclyl, or any subgroup thereof, wherein the C₁₋₆alkyl, the C₃₋₁₂cycloalkyl, the C₆₋₁₀aryl, the C₁₋₆alkyl-C₆₋₁₀aryl, or the C₅₋₁₅heterocyclyl is optionally substituted by 1, 2, or 3 substitutents independently selected from: halogen, CN, NH₂, OH, COOH, OC₁₋₆alkyl, CH₂OH, SO₂H, S(═O), C₂₋₆alkenyl, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂ S(═O)C₁₋₆alkyl-R^{a}, S(═O)NHC₁₋₆alkyl-R^{a}, S(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-Ia, N(C₁₋₆alkyl-R^{a})₂, NHC(═O)C₁₋₆alkyl, C₆₋₁₀aryl-R^{a}, OC₆₋₁₀aryl-R^{a}, C(═O)C₆₋₁₀aryl-R^{a}, C(═O)OC₆₋₁₀aryl-R^{a}, C(═O)NHC₆₋₁₀aryl-R^{a}, C(═O)N(C₆₋₁₀aryl-R^{a})₂, S(═O)C₆₋₁₀aryl-R^{a}, S(═O)NHC₆₋₁₀aryl-R^{a}, S(═O)N(C₆₋₁₀aryl-R^{a})₂, SO₂C₆₋₁₀aryl-R^{a}, SO₂NHC₆₋₁₀aryl-R^{a}, SO₂N(C₆₋₁₀aryl-R^{a})₂, NH(C₆₋₁₀aryl)-R^{a}, N(C₆₋₁₀aryl-R^{a})₂, NC(═O)C₆₋₁₀aryl, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NHC₅₋₆heterocyclyl-R^{a}, C(═O)N(C₅₋₆heterocyclyl-R^{a})₂, S(═O)C₅₋₆heterocyclyl-IV, S(═O)NHC₅₋₆heterocyclyl-R^{a}, S(═O)N(C₅₋₆heterocyclyl-R)₂, SO₂C₅₋₆heterocyclyl-R^{a}, SO₂NHC₅₋₆heterocyclyl-R^{a}, SO₂N(C₅₋₆heterocyclyl-R^{a})₂, NH(C₅₋₆heterocyclyl-R^{a}), N(C₅₋₆heterocyclyl-R^{a})₂, NHC(═O)C₅₋₆heterocyclyl, SO₂R^{a}, S(═O)R^{a}, N(C₁₋₆alkyl-R^{a})(C₆₋₁₀aryl-W), N(C₁₋₆alkyl-R^{a})(C₆₋₁₀heteroaryl-R^{a}), N(C₆₋₁₀aryl-R^{a})(C₆₋₁₀heteroaryl-R^{a}), C(═O)(C₁₋₆alkyl-R^{a})(C₆₋₁₀aryl-R^{a}), C(═O)(C₁₋₆alkyl-R^{a})(C₆₋₁₀heteroaryl-R^{a}), C(═O)(C₆₋₁₀aryl-R^{a})(C₆₋₁₀heteroaryl-R^{a}), C(═O)O(C₁₋₆alkyl-R^{a})(C₆₋₁₀aryl-R^{a}), C(═O)O(C₁₋₆alkyl-R^{a})(C₆₋₁₀heteroaryl-R^{a}), C(═O)O(C₆₋₁₀aryl-R^{a})(C₆₋₁₀heteroaryl-R^{a}), S(═O)(C₁₋₆alkyl-R^{a})(C₆₋₁₀aryl-R^{a}), S(═O)(C₁₋₆alkyl-R^{a})(C₆₋₁₀heteroaryl-R^{a}), S(═O)(C₆₋₁₀aryl-R^{a})(C₆₋₁₀heteroaryl-R^{a}), SO₂(C₁₋₆alkyl-R^{a})(C₆₋₁₀aryl-R^{a}), SO₂(C₁₋₆alkyl-R^{a})(C₆₋₁₀heteroaryl-R^{a}), or SO₂(C₆₋₁₀aryl-R^{a})(C₆₋₁₀heteroaryl-R^{a}), or any subgroup thereof. In some embodiments, R¹ is, independently, selected from H, halogen, C₆aryl, or C₅₋₆heterocyclyl wherein the C₆aryl, or C₅₋₆heterocyclyl is optionally substituted by 1, 2, or 3 substituents, independently, selected from: halogen, OH, NH₂, CN, C(═O)NH₂, C₁₋₆alkyl, OC₁₋₆alkyl, C₁₋₄alkylOH, C₁₋₄alkylOC₁₋₃alkyl, CH₂OH, SO₂H, SO₂NHC(CH₃)₃, SO₂C₁₋₆alkyl, SO₂NHC₁₋₆alkyl, OC₁₋₃alkylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₅₋₆heterocyclyl, OC₅₋₆aryl, —C₆aryl-OC₁₋₄alkyl or OC₁₋₆alkyl-C₅₋₆aryl. In some embodiments, R¹ is C₆₋₁₀aryl or C₅₋₁₅heterocyclyl, wherein each the aryl and heterocyclyl is optionally substituted by 1 or 2 substituents independently selected from: halogen, CN, C₁₋₄alkyl, C₁₋₄haloalkyl, OC₁₋₄alkyl, OC₁₋₄haloalkyl, —C(O)H, COOH, OC₁₋₄alkyl-C₆₋₁₀aryl, OH, NHC(═O)C₁₋₄alkyl and —C₆aryl-OC₁₋₄alkyl. In some embodiments, R¹ is C₆₋₁₀aryl optionally substituted by 1 or 2 substituents independently selected from: OC₁₋₄alkyl and —C₆aryl-OC₁₋₄alkyl.

In some embodiments, each R^{a} is, independently, selected from H, halogen, CN, NH₂, OH, C₁₋₆alkyl, OC₁₋₆alkyl, C(═O)C₁₋₆alkyl, C(═O)OC₁₋₆alkyl, C(═O)NH₂, C(═O)NHC₁₋₆alkyl, C(—O)N(C₁₋₆alkyl)₂, SOC₁₋₆alkyl, SONHC₁₋₆alkyl, SON(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆allyl)₂, NC(═O)C₁₋₆alkyl, C₅₋₆aryl, OC₅₋₆aryl, C(═O)C₅₋₆aryl, C(═O)OC₅₋₆aryl, C(—O)NH₂, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NC(═O)C₅₋₆aryl, C₅₋₆heterocyclyl, OC₅₋₆heterocyclyl, C(═O)C₅₋₆heterocyclyl, C(═O)OC₅₋₆heterocyclyl, C(═O)NH₂, C(═O)NHC₅₋₆heterocyclyl, C(═O)N(C₅₋₆heterocyclyl)₂, S(═O)C₅₋₆heterocyclyl, S(═O)NHC₅₋₆heterocyclyl, S(═O)N(C₅₋₆heterocyclyl)₂, SO₂NHC₅₋₆heterocyclyl, SO₂N(C₅₋₆heterocyclyl)₂, NH(C₅₋₆heterocyclyl), N(C₅₋₆heterocyclyl)₂, NC(═O)C₅₋₆heterocyclyl, C(═O)NHC₁₋₆alkylC₅₋₆aryl, NR^{b}R^{b}, C(═O)R^{b}, C(═O)NR^{b}R^{b}, OC(═O)NR^{b}R^{b}, S(═O)R^{b}, S(═O)NR^{b}R^{b}, or SO₂NR^{b}R^{b}, or any subgroup thereof.

In some embodiments, each R^{b} is, independently, selected from H, C₁₋₆alkyl, C₅₋₆aryl, or C₅₋₆heterocyclyl, or any subgroup thereof.

In some embodiments, each V is, independently, selected from NH, O, S, S(═O), SO₂, NHS(═O), NHSO₂, S(═O)NH, SO₂NH, NHC(═O), C(═O)NH, NR^{a}SO₂, NR^{a}S(═O), NR^{a}C(O), C(O)NR^{a}, S(O)₂NR^{a}, S(═O)NR^{a}, OC₁₋₆alkylenyl, C₂₋₆alkenylenyl or C₁₋₆alkylenyl, or any subgroup thereof wherein the OC₁₋₆alkylenyl, C₂₋₆alkenylenyl, and C₁₋₆alkylenyl is optionally substituted by 1, 2, or 3 substitutents independently selected from R^{a}, or any subgroup thereof.

In some embodiments, X and Y are each independently selected from NH, O, S, S(═O), SO₂, NHS(═O), NHSO₂, S(═O)NH, SO₂NH, NHC(═O), C(═O)NH, NR^{a}SO₂, NR^{a}S(═O), NR^{a}C(O), C(O)NR^{a}, S(O)₂NR^{a}, S(═O)NR^{a}, or C₁₋₆alkyl, or any subgroup thereof, wherein the C₁₋₆alkyl is optionally substituted by 1, 2, or 3 substituents independently selected from R^{a}, or any subgroup thereof.

In some embodiments, Z is

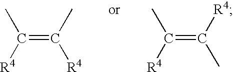

In some embodiments, m is 0, 1, 2 or 3, or any subgroup thereof.

In some embodiments, n, q, r, and u are each, independently, 0 or 1.

In some embodiments, s is 1 or 2.

In some embodiments, R² is selected from H, halogen, C₁₋₆alkyl, C₃₋₁₂cycloalkyl, C₆₋₁₀aryl, C₁₋₆alkyl-C₆₋₁₀aryl, C₅₋₁₀heterocyclyl, or C₁₋₆alkyl-C₅₋₁₀heterocyclyl, or any subgroup thereof, wherein the C₁₋₆alkyl, C₃₋₁₂cycloalkyl, C₆₋₁₀aryl, C₁₋₆alkyl-C₆₋₁₀aryl, C₅₋₁₀heterocyclyl, and C₁₋₆alkyl-C₅₋₁₀heterocyclyl is optionally substituted by 1, 2, or 3 substitutents independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂ S(═O)C₁₋₆alkyl-R^{a}, S(═O)NHC₁₋₆alkyl-R^{a}, S(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-R^{a}, N(C₁₋₆alkyl-R^{a})₂, NHC(═O)C₁₋₆alkyl, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl-R^{a})₂, S(═O)C₅₋₆aryl-R^{a}, S(═O)NHC₅₋₆aryl-R^{a}, S(═O)N(C₅₋₆aryl-R^{a})₂, SO₂C₅₋₆aryl-R^{a}, SO₂NHC₅₋₆aryl-R^{a}, SO₂N(C₅₋₆alkyl-R^{a})₂, NH(C₅₋₆aryl)-R^{a}, N(C₅₋₆aryl-R^{a})₂, NHC(═O)C₅₋₆aryl, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-IV, C(═O)NH₂, C(═O)NHC₅₋₆heterocyclyl-IV, C(═O)N(C₅₋₆heterocyclyl-R^{a})₂, SO₂C₅₋₆heterocyclyl-R^{a}, SO₂NHC₅₋₆heterocyclyl-R^{a}, SO₂N(C₅₋₆heterocyclyl-R^{a})₂ S(═O)C₅₋₆heterocyclyl-R^{a}, S(═O)NHC₅₋₆heterocyclyl-R^{a}, S(═O)N(C₅₋₆heterocyclyl-R^{a})₂, NH(C₅₋₆heterocyclyl)-R^{a}, N(C₅₋₆heterocyclyl-R^{a})₂, or NHC(═O)C₅₋₆heterocyclyl, or any subgroup thereof. In some embodiments, R² is C₁₋₃alkyl.

In some embodiments, R³ is selected from R¹, C₁₋₆alkylR^{c}, C₁₋₆alkylR^{c}, C₁₋₆alkylOR^{c}, C₁₋₆alkylSR^{c}, C₁₋₆alkylNHC₁₋₆alkylC₅₋₆arylR^{d}, C₁₋₆alkylNHC₆₋₁₀arylR^{d}, C₁₋₆alkylNHC(O)C₆₋₁₀arylR^{d}, C₁₋₆alkylOC₁₋₆alkylC₅₋₆aryl, C₁₋₆alkylSC₁₋₆alkylC₅₋₆arylR^{d}, C₁₋₆alkylC₅₋₉heterocyclylR^{d}, C₁₋₆alkylC₃₋₉cycloalkylR^{d}, C₁₋₆alkylNHC₁₋₆alkylC₅₋₉heterocyclylR^{d}, C₁₋₆alkylNHC₅₋₉heterocyclyl(R^{d})ₜ, C₁₋₆alkylNHC(O)C₅₋₉heterocyclylR^{d}, C₁₋₆alkylOC₁₋₆alkylC₅₋₉heterocyclylR^{d}, C₁₋₆alkylSC₁₋₆alkylC₅₋₉heterocyclylR^{d}, C₁₋₆alkylNHC₁₋₆alkylC₃₋₉cycloalkylR^{d}, C₁₋₆alkylOC₁₋₆alkylC₃₋₉cycloalkylR^{d}, or C₁₋₆alkylSC₁₋₆alkylC₃₋₉cycloalkylR^{d}. In some embodiments, R³ is selected from H, C₁₋₆alkyl, C₁₋₆alkylNR^{c}R^{c}, C₁₋₆alkylOR^{c}, C₁₋₆alkylNHC₁₋₆alkylC₆₋₁₀arylR^{d}, C₁₋₆alkylNHC(O)C₆₋₁₀arylR^{d}, C₁₋₆alkylOC₁₋₆alkylC₅₋₆arylR^{d}, C₁₋₆alkylC₆₋₁₀arylR^{d}, C₁₋₆alkylC₅₋₉heterocyclyl, or C₁₋₆alkylC₃₋₉cycloalkylR^{d}. In some embodiments, R³ is selected from H, C₁₋₆alkyl, C₁₋₆alkylNR^{c}R^{c}, or C₁₋₆alkyl-C₅₋₉heterocyclylR^{d}. In some embodiments, R³ is H or C₁₋₃alkyl.

In some embodiments, R³ is C₁₋₃alkyl.

In some embodiments, each R⁴ is, independently, selected from H, halogen, C₁₋₆alkyl, C₃₋₁₂cycloalkyl, C₆₋₁₀aryl, C₁₋₆alkyl-C₆₋₁₀aryl, C₅₋₁₀heterocyclyl, or C₁₋₆alkyl-C₅₋₁₀heterocyclyl, or any subgroup thereof, wherein the C₁₋₆alkyl, C₃₋₁₂cycloalkyl, C₆₋₁₀aryl, C₁₋₆alkyl-C₆₋₁₀aryl, C₅₋₁₀heterocyclyl, and C₁₋₆alkyl-C₅₋₁₀heterocyclyl is optionally substituted by 1, 2, or 3 substitutents independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-IV)₂ S(═O)C₁₋₆alkyl-R^{a}, S(═O)NHC₁₋₆alkyl-IV, S(═O)N(C₁₋₆alkyl-IV)₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-IV, N(C₁₋₆alkyl-R^{a})₂, or NHC(═O)C₁₋₆alkyl, or any subgroup thereof. In some embodiments, each R⁴ is H.

In some embodiments, t is 0, 1, 2, 3, 4 or 5.

In some embodiments, each R^{c} is, independently, selected from H, C(═O)C₁₋₄alkyl, C(═O)C₁₋₄alkylOC₁₋₄alkyl, C(═O)C₁₋₄alkylC(═O)OC₁₋₄alkyl, C(═O)C₁₋₄alkylC(═O)OH, C(═O)C₁₋₄alkylOC(═O)C₁₋₄alkyl, C₅₋₆arylR^{d}, C₅₋₉heterocyclylR^{d}, C₃₋₉cycloalkylR^{d}, C(═O)C₅₋₆arylR^{d}, C(═O)C₅₋₉heterocyclylR^{d}, C(═O)C₃₋₉cycloalkylR^{d}, C₁₋₄alkyl-C₅₋₆arylR^{d}. C₁₋₄alkyl-C₅₋₉heterocyclylR^{d}, or C₁₋₄alkyl-C₃₋₉cycloalkylR^{d}, or any subgroup thereof.

In some embodiments, R^{d} is selected from H, C₁₋₃alkyl, NH₂, OH, COOH, OC₁₋₃alkyl, or OC₁₋₃alkylOH, or any subgroup thereof.

When the compound has formula Ib, however, and W is N, u is 1, and R³ is H, then [R¹-(V)ₙ]ₘ-Q is other than polyC₁₋₄alkyl substituted cyclohexenyl or a nitro substituted furyl.

In some embodiments, the compound has the structure of formula Ia.

In some embodiments, the compound has the structure of formula Ib.

In some embodiments, -[X]_{q}-[Y]ᵣ- is OC₁₋₃alkyl, N(C₁₋₃alkyl)C₁₋₃alkyl, C₁₋₃alkylOC₁₋₃alkyl, C₁₋₃alkylN(H)C₁₋₃alkyl or C₁₋₃alkyl optionally substituted by OH. In some embodiments, -[X]_{q}-[Y]ᵣ- is OC₁₋₃alkyl. In some embodiments, -[X]_{q}-[Y]ᵣ- is absent.

In some embodiments, q is 0, r is 0, and each R⁴ is H.

In some embodiments, m is 1, V is S, n is 0 or 1, and R¹ is C₆₋₁₀aryl or C₅₋₁₅heterocyclyl, wherein each the aryl and heterocyclyl is optionally substituted by 1 or 2 substituents independently selected from: halogen, CN, C₁₋₄alkyl, C₁₋₄haloalkyl, OC₁₋₄alkyl, OC₁₋₄haloalkyl, —C(O)H, COOH, OC₁₋₄alkyl-C₆₋₁₀aryl, OH, NHC(═O)C₁₋₄alkyl and —C₆aryl-OC₁₋₄alkyl.

In some embodiments, m is 1, n is 0, and R¹ is C₆₋₁₀aryl, wherein the aryl is optionally substituted by 1 or 2 substituents independently selected from: halogen, CN, C₁₋₄alkyl, C₁₋₄haloalkyl, OC₁₋₄alkyl, OC₁₋₄haloalkyl, —C(O)H, COOH, OC₁₋₄alkyl-C₆₋₁₀aryl, OH, NHC(═O)C₁₋₄alkyl and —C₆aryl-OC₁₋₄alkyl.

In some embodiments, R¹ is, independently, selected from H, halogen, C₆aryl, or C₅₋₆heterocyclyl wherein the C₆aryl, or C₅₋₆heterocyclyl is optionally substituted by 1, 2, or 3 substituents, independently, selected from: halogen, OH, NH₂, CN, C(—O)NH₂, C₁₋₆alkyl, OC₁₋₆alkyl, C₁₋₄alkylOH, C₁₋₄alkylOC₁₋₃alkyl, CH₂OH, SO₂H, SO₂NHC(CH₃)₃, SO₂C₁₋₆alkyl, SO₂NHC₁₋₆alkyl, OC₁₋₃alkylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₅₋₆heterocyclyl, OC₅₋₆aryl, —C₆aryl-OC₁₋₄alkyl or OC₁₋₆alkyl-C₅₋₆aryl; R² is H or C₁₋₆alkyl; R³ is H or C₁₋₃alkyl; and each R⁴ is H.

In some embodiments, Q is C₆aryl or C₅₋₉heterocyclyl; W is N; R¹ is, independently, selected from H, halogen, C₆aryl, or C₅₋₆heterocyclyl wherein the C₆aryl, or C₅₋₆heterocyclyl is optionally substituted by 1, 2, or 3 substituents, independently, selected from: halogen, OH, NH₂, CN, C(═O)NH₂, C₁₋₆alkyl, OC₁₋₆alkyl, C₁₋₄alkylOH, C₁₋₄alkylOC₁₋₃alkyl, CH₂OH, SO₂H, SO₂NHC(CH₃)₃, SO₂C₁₋₆alkyl, SO₂NHC₁₋₆alkyl, OC₁₋₃alkylOC₁₋₃alkyl, OC₁₋₃alkylOH, OC₁₋₃alkylOC(═O)C₁₋₃alkyl, C(═O)C₁₋₆alkyl, C(—O)OC₁₋₆alkyl, C(═O)NH₂, C₅₋₆heterocyclyl, OC₅₋₆aryl, —C₆aryl-OC₁₋₄alkyl or OC₁₋₆alkyl-C₅₋₆aryl; and R² is C₁₋₃alkyl.

In some embodiments, Q is C₆₋₁₀aryl; W is N; -[X]_{q}-[Y]ᵣ- is OC₁₋₃alkyl; m is 1; n is 0; and R¹ is C₆₋₁₀aryl optionally substituted by 1 or 2 substituents independently selected from: OC₁₋₄alkyl and —C₆aryl-OC₁₋₄alkyl.

In some embodiments, Q is C₃₋₁₀cycloalkenyl; W is N; -[X]_{q}-[Y]ᵣ- is absent; m is 1; n is 0; and R¹ is C₆₋₁₀aryl optionally substituted by 1 or 2 substituents independently selected from: OC₁₋₄alkyl and —C₆aryl-OC₁₋₄alkyl.

In some embodiments, Q is C₆₋₁₀aryl, C₃₋₁₀cycloalkyl or C₃₋₁₀cycloalkenyl; W is N; -[X]_{q} [Y]ᵣ- is OC₁₋₃alkyl, N(C₁₋₃alkyl)C₁₋₃alkyl, C₁₋₃alkylOC₁₋₃alkyl, C₁₋₃alkylN(H)C₁₋₃alkyl or C₁₋₃alkyl optionally substituted by OH; m is 1; V is S; n is 0 or 1; and R¹ is C₆₋₁₀aryl or C₅₋₁₅ heterocyclyl, wherein each the aryl and heterocyclyl is optionally substituted by 1 or 2 substituents independently selected from: halogen, CN, C₁₋₄alkyl, C₁₋₄haloalkyl, OC₁₋₄alkyl, OC₁₋₄haloalkyl, —C(O)H, COOH, OC₁₋₄alkyl-C₆₋₁₀aryl, OH, NHC(—O)C₁₋₄alkyl and —C₆aryl-OC₁₋₄alkyl.

In some embodiments, the compound is
2-amino-6-[[3-(3-methoxyphenyl)phenoxy]methyl]-3-methyl-3H-pyrimidin-4-one, or
2-amino-6-[2-[3-(3-methoxyphenyl)phenyl]-3-bicyclo[2.2.1]hept-5-enyl]-3-methyl-3H-pyrimidin-4-one;
   or a pharmaceutically acceptable salt, tautomer, or in vivo-hydrolysable precursor thereof.

Compounds of the present invention also include pharmaceutically acceptable salts, tautomers and in vivo-hydrolysable precursors of the compounds of formula Ia or Ib. Compounds of the invention further include hydrates and solvates.

Compounds of the invention can be used as medicaments. In some embodiments, the present invention provides compounds of formula Ia or Ib, or pharmaceutically acceptable salts, tautomers or in vivo-hydrolysable precursors thereof, for use as medicaments. In some embodiments, the present invention provides compounds described herein for use as as medicaments for treating or preventing an Aβ-related pathology. In some further embodiments, the Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In some embodiments, the present invention provides compounds of formula Ia or Ib, or pharmaceutically acceptable salts, tautomers or in vivo-hydrolysable precursors thereof, in the manufacture of a medicament for the treatment or prophylaxis of Aβ-related pathologies. In some further embodiments, the Aβ-related pathologies include such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method of inhibiting activity of BACE comprising contacting the BACE with a compound of the present invention. BACE is thought to represent the major β-secretase activity, and is considered to be the rate-limiting step in the production of amyloid-β-protein (Aβ). Thus, inhibiting BACE through inhibitors such as the compounds provided herein would be useful to inhibit the deposition of Aβ and portions thereof. Because the deposition of Aβ and portions thereof is linked to diseases such as Alzheimer Disease, BACE is an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method for the treatment of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, comprising administering to a mammal (including human) a therapeutically effective amount of a compound of formula Ia or Ib, or a pharmaceutically acceptable salt, tautomer or in vivo-hydrolysable precursor thereof.

In some embodiments, the present invention provides a method for the prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration comprising administering to a mammal (including human) a therapeutically effective amount of a compound of formula Ia or Ib or a pharmaceutically acceptable salt, tautomer or in vivo-hydrolysable precursors.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) a compound of formula Ia or Ib or a pharmaceutically acceptable salt, tautomer or in vivo-hydrolysable precursors and a cognitive and/or memory enhancing agent.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) a compound of formula Ia or Ib or a pharmaceutically acceptable salt, tautomer or in vivo-hydrolysable precursors thereof wherein constituent members are provided herein, and a choline esterase inhibitor or anti-inflammatory agent.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, or any other disease, disorder, or condition described herein, by administering to a mammal (including human) a compound of the present invention, and an atypical antipsychotic agent. Atypical antipsychotic agents includes, but not limited to, Olanzapine (marketed as Zyprexa), Aripiprazole (marketed as Abilify), Risperidone (marketed as Risperdal), Quetiapine (marketed as Seroquel), Clozapine (marketed as Clozaril), Ziprasidone (marketed as Geodon) and Olanzapine/Fluoxetine (marketed as Symbyax).

In some embodiments, the mammal or human being treated with a compound of the present invention, has been diagnosed with a particular disease or disorder, such as those described herein. In these cases, the mammal or human being treated is in need of such treatment. Diagnosis, however, need not be previously performed.

The anti-dementia treatment defined herein may be applied as a sole therapy or may involve, in addition to the compound of the invention, conventional chemotherapy. Such chemotherapy may include one or more of the following categories of agents: acetyl cholinesterase inhibitors, anti-inflammatory agents, cognitive and/or memory enhancing agents or atypical antipsychotic agents.

Such conjoint treatment may be achieved by way of the simultaneous, sequential or separate dosing of the individual components of the treatment. Such combination products employ the compounds of this invention.

Cognitive enhancing agents memory enhancing agents and choline esterase inhibitors includes, but not limited to, onepezil (Aricept), galantamine (Reminyl or Razadyne), rivastigmine (Exelon), tacrine (Cognex) and memantine (Namenda, Axura or Ebixa).

The present invention also includes pharmaceutical compositions which contain, as the active ingredient, one or more of the compounds of the invention herein together with at least one pharmaceutically acceptable carrier, diluent or excipient.

When used for pharmaceutical compositions, medicaments, manufacture of a medicament, inhibiting activity of BACE, or treating or preventing Aβ-related pathologies, compounds of the present invention include the compounds of formula Ia or Ib, and pharmaceutically acceptable salts, tautomers and in vivo-hydrolysable precursors thereof. Compounds of the present invention further include hydrates and solvates.

The definitions set forth in this application are intended to clarify terms used throughout this application. The term “herein” means the entire application.

As used in this application, the term “optionally substituted,” as used herein, means that substitution is optional and therefore it is possible for the designated atom or moiety to be unsubstituted. In the event a substitution is desired then such substitution means that any number of hydrogens on the designated atom or moiety is replaced with a selection from the indicated group, provided that the normal valency of the designated atom or moiety is not exceeded, and that the substitution results in a stable compound. For example, if a methyl group (i.e., CH₃) is optionally substituted, then 3 hydrogens on the carbon atom can be replaced. Examples of suitable substituents include, but are not limited to: halogen, CN, NH₂, OH, SO, SO₂, COOH, OC₁₋₆alkyl, CH₂OH, SO₂H, C₁₋₆alkyl, OC₁₋₆alkyl, C(═O)C₁₋₆alkyl, C(═O)OC₁₋₆alkyl, C(═O)NH₂, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(═O)C₁₋₆alkyl, NC(═O)(C₁₋₆alkyl)₂, C₅₋₆aryl, OC₅₋₆aryl, C(═O)C₅₋₆aryl, C(═O)OC₅₋₆aryl, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NC(═O)C₅₋₆aryl, NC(═O)(C₅₋₆aryl)₂, C₅₋₆heterocyclyl, OC₅₋₆heterocyclyl, C(═O)C₅₋₆heterocyclyl, C(═O)OC₅₋₆heterocyclyl, C(═O)NHC₅₋₆heterocyclyl, C(═O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NC(═O)C₅₋₆heterocyclyl, NC(═O)(C₅₋₆heterocyclyl)₂.

A variety of compounds in the present invention may exist in particular geometric or stereoisomeric forms. The present invention takes into account all such compounds, including cis- and trans isomers, R- and S-enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

As used herein, “alkyl”, “alkylenyl” or “alkylene” used alone or as a suffix or prefix, is intended to include both branched and straight-chain saturated aliphatic hydrocarbon groups having from 1 to 12 carbon atoms or if a specified number of carbon atoms is provided then that specific number would be intended. For example “C₁₋₆alkyl” denotes alkyl having 1, 2, 3, 4, 5 or 6 carbon atoms. Examples of alkyl include, but are not limited to, methyl, ethyl, n-propyl, i-propyl, n-butyl, i-butyl, sec-butyl, t-butyl, pentyl, and hexyl. As used herein, “C₁₋₃alkyl”, whether a terminal substituent or an alkylene (or alkylenyl) group linking two substituents, is understood to specifically include both branched and straight-chain methyl, ethyl, and propyl.

As used herein, “alkenyl” refers to an alkyl group having one or more double carbon-carbon bonds. Example alkenyl groups include ethenyl, propenyl, cyclohexenyl, and the like. The term “alkenylenyl” refers to a divalent linking alkenyl group.

As used herein, “alkynyl” refers to an alkyl group having one or more triple carbon-carbon bonds. Example alkynyl groups include ethynyl, propynyl, and the like. The term “alkynylenyl” refers to a divalent linking alkynyl group.

As used herein, “aromatic” refers to hydrocarbyl groups having one or more polyunsaturated carbon rings having aromatic characters, (e.g., 4n+2 delocalized electrons) and comprising up to about 14 carbon atoms.

As used herein, the term “aryl” refers to an aromatic ring structure made up of from 5 to 14 carbon atoms. Ring structures containing 5, 6, 7 and 8 carbon atoms would be single-ring aromatic groups, for example, phenyl. Ring structures containing 8, 9, 10, 11, 12, 13, or 14 would be a polycyclic moiety in which at least one carbon is common to any two adjoining rings therein (for example, the rings are “fused rings”), for example naphthyl. The aromatic ring can be substituted at one or more ring positions with such substituents as described above. The term “aryl” also includes polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls or cycloalkynyls. The terms ortho, meta and para apply to 1,2-, 1,3- and 1,4-disubstituted benzenes, respectively. For example, the names 1,2-dimethylbenzene and ortho-dimethylbenzene are synonymous.

As used herein, “cycloalkyl” refers to non-aromatic cyclic hydrocarbons including cyclized alkyl, alkenyl, and alkynyl groups, having the specified number of carbon atoms. Cycloalkyl groups can include mono- or polycyclic (e.g., having 2, 3 or 4 fused or bridged rings) groups. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (i.e., having a bond in common with) to the cycloalkyl ring, for example, benzo derivatives of cyclopentane (i.e., indanyl), cyclopentene, cyclohexane, and the like. The term “cycloalkyl” further includes saturated ring groups, having the specified number of carbon atoms. These may include fused or bridged polycyclic systems. Preferred cycloalkyls have from 3 to 10 carbon atoms in their ring structure, and more preferably have 3, 4, 5, and 6 carbons in the ring structure. For example, “C₃₋₆ cycloalkyl” denotes such groups as cyclopropyl, cyclobutyl, cyclopentyl, or cyclohexyl.

As used herein, “cycloalkenyl” refers to ring-containing hydrocarbyl groups having at least one carbon-carbon double bond in the ring, and having from 3 to 12 carbons atoms.

As used herein, “halo” or “halogen” refers to fluoro, chloro, bromo, and iodo. “Counterion” is used to represent a small, negatively or positively charged species such as chloride (Cl⁻), bromide (Br⁻), hydroxide (OH⁻), acetate (CH₃COO⁻), sulfate (SO₄²⁻), tosylate (CH₃-phenyl-SO₃⁻), benezensulfonate (phenyl-SO₃⁻), sodium ion (Na⁺), potassium (K⁺), ammonium (NH₄⁺), and the like.

As used herein, the term “heterocyclyl” or “heterocyclic” or “heterocycle” refers to a ring-containing monovalent and divalent structures having one or more heteroatoms, independently selected from N, O and S, as part of the ring structure and comprising from 3 to 20 atoms in the rings, more preferably 3- to 7-membered rings. The number of ring-forming atoms in heterocyclyl are given in ranges herein. For example, C₅₋₁₀ heterocyclyl refers to a ring structure comprising from 5 to 10 ring-forming atoms wherein at least one of the ring-forming atoms is N, O or S. Heterocyclic groups may be saturated or partially saturated or unsaturated, containing one or more double bonds, and heterocyclic groups may contain more than one ring as in the case of polycyclic systems. The heterocyclic rings described herein may be substituted on carbon or on a heteroatom atom if the resulting compound is stable. If specifically noted, nitrogen in the heterocyclyl may optionally be quaternized. It is understood that when the total number of S and O atoms in the heterocyclyl exceeds 1, then these heteroatoms are not adjacent to one another.

Examples of heterocyclyls include, but are not limited to, 1H-indazole, 2-pyrrolidonyl, 2H, 6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azabicyclo, azetidine, azepane, aziridine, azocinyl, benzimidazolyl, benzodioxol, benzofuranyl, benzothiofuranyl, benzothiophenyl, benzoxazolyl, benzthiazolyl, benzotriazolyl, benzotetrazolyl, benzisoxazolyl, benzisothiazolyl, benzimidazalonyl, carbazolyl, 4aH-carbazolyl, b-carbolinyl, chromanyl, chromenyl, cinnolinyl, diazepane, decahydroquinolinyl, 2H,6H-1,5,2-dithiazinyl, dioxolane, furyl, 2,3-dihydrofuran, 2,5-dihydrofuran, dihydrofuro[2,3-b]tetrahydrofuran, furanyl, furazanyl, homopiperidinyl, imidazolidine, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxirane, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, purinyl, pyranyl, pyrrolidinyl, pyrroline, pyrrolidine, pyrazinyl, pyrazolidinyl, pyrazolinyl, pyrazolyl, pyridazinyl, pyridooxazole, pyridoimidazole, pyridothiazole, pyridinyl, N-oxide-pyridinyl, pyridyl, pyrimidinyl, pyrrolidinyl, pyrrolidinyl dione, pyrrolinyl, pyrrolyl, pyridine, quinazolinyl, quinolinyl, 4H-quinolizinyl, quinoxalinyl, quinuclidinyl, carbolinyl, tetrahydrofuranyl, tetramethylpiperidinyl, tetrahydroquinoline, tetrahydroisoquinolinyl, thiophane, thiotetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiopheneyl, thiirane, triazinyl, 1,2,3-triazolyl, 1,2,4-triazolyl, 1,2,5-triazolyl, 1,3,4-triazolyl, xanthenyl.

As used herein, “heteroaryl” refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 4 to about 14, 3 to about 7, or 5 to 6 ring-forming atoms. In some embodiments, the heteroaryl group has 1 to about 4, 1 to about 3, or 1 to 2 heteroatoms. In some embodiments, the heteroaryl group has 1 heteroatom.

As used herein, “alkoxy” or “alkyloxy” represents an alkyl group as defined above with the indicated number of carbon atoms attached through an oxygen bridge. Examples of alkoxy include, but are not limited to, methoxy, ethoxy, n-propoxy, isopropoxy, n-butoxy, isobutoxy, t-butoxy, n-pentoxy, isopentoxy, cyclopropylmethoxy, allyloxy and propargyloxy. Similarly, “alkylthio” or “thioalkoxy” represent an alkyl group as defined above with the indicated number of carbon atoms attached through a sulphur bridge.

As used herein, the term “carbonyl” is art recognized and includes such moieties as can be represented by the general formula:

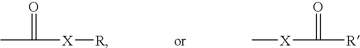

wherein X is a bond or represents an oxygen or sulfur, and R represents a hydrogen, an alkyl, an alkenyl, —(CH₂)ₘ—R″ or a pharmaceutically acceptable salt, R¹ represents a hydrogen, an alkyl, an alkenyl or —(CH₂)ₘ—R″, where m is an integer less than or equal to ten, and R″ is alkyl, cycloalkyl, alkenyl, aryl, or heteroaryl. Where X is an oxygen and R and R′ is not hydrogen, the formula represents an “ester”. Where X is an oxygen, and R is as defined above, the moiety is referred to herein as a carboxyl group, and particularly when R′ is a hydrogen, the formula represents a “carboxylic acid.” Where X is oxygen, and R′ is a hydrogen, the formula represents a “formate.” In general, where the oxygen atom of the above formula is replaced by sulfur, the formula represents a “thiolcarbonyl” group. Where X is a sulfur and R and R′ is not hydrogen, the formula represents a “thiolester.” Where X is sulfur and R is hydrogen, the formula represents a “thiolcarboxylic acid.” Where X is sulfur and R′ is hydrogen, the formula represents a “thiolformate.” On the other hand, where X is a bond, and R is not a hydrogen, the above formula represents a “ketone” group. Where X is a bond, and R is hydrogen, the above formula is represents an “aldehyde” group.

As used herein, the term “sulfonyl” refers to a moiety that can be represented by the general formula:

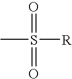

wherein R is represented by but not limited to hydrogen, alkyl, cycloalkyl, alkenyl, aryl, heteroaryl, aralkyl, or heteroaralkyl.

As used herein, some substituents are described in a combination of two or more groups. For example, the expression of “C(═O)C₃₋₉cycloalkylR^{d}” is meant to refer to a structure:

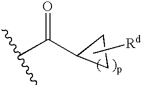

wherein p is 1, 2, 3, 4, 5, 6 or 7 (i.e., C₃₋₉cycloalkyl); the C₃₋₉cycloalkyl is substituted by R^{d}; and the point of attachment of the “C(═O)C₃₋₉cycloalkylR^{d}” is through the carbon atom of the carbonyl group, which is on the left of the expression.

As used herein, the phrase “protecting group” means temporary substituents which protect a potentially reactive functional group from undesired chemical transformations. Examples of such protecting groups include esters of carboxylic acids, silyl ethers of alcohols, and acetals and ketals of aldehydes and ketones respectively. The field of protecting group chemistry has been reviewed (Greene, T. W.; Wuts, P.G.M. Protective Groups in Organic Synthesis, 3^{rd} ed.; Wiley: New York, 1999).

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof (i.e., also include counterions). Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, phosphoric, and the like; and the salts prepared from organic acids such as lactic, maleic, citric, benzoic, methanesulfonic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; nonaqueous media like ether, ethyl acetate, ethanol, isopropanol, or acetonitrile can be used.

As used herein, “in vivo hydrolysable precursors” means an in vivo hydroysable (or cleavable) ester of a compound of formula Ia or Ib that contains a carboxy or a hydroxy group. For example amino acid esters, C₁₋₆ alkoxymethyl esters like methoxymethyl; C₁₋₆alkanoyloxymethyl esters like pivaloyloxymethyl; C₃₋₈cycloalkoxycarbonyloxy C₁₋₆alkyl esters like 1-cyclohexylcarbonyloxyethyl, acetoxymethoxy, or phosphoramidic cyclic esters.

As used herein, “tautomer” means other structural isomers that exist in equilibrium resulting from the migration of a hydrogen atom. For example, keto-enol tautomerism where the resulting compound has the properties of both a ketone and an unsaturated alcohol.

As used herein “stable compound” and “stable structure” are meant to indicate a compound that is sufficiently robust to survive isolation to a useful degree of purity from a reaction mixture, and formulation into an efficacious therapeutic agent.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled compound” is a compound that has incorporated at least one radionuclide. In some embodiments the radionuclide is selected from the group consisting of ³H, ¹⁴C, ¹²⁵I, ³⁵S and ⁸²Br.

The antidementia treatment defined herein may be applied as a sole therapy or may involve, in addition to the compound of the invention, conventional chemotherapy.

Such conjoint treatment may be achieved by way of the simultaneous, sequential or separate dosing of the individual components of the treatment. Such combination products employ the compounds of this invention.

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of dementia is an amount sufficient to symptomatically relieve in a warm-blooded animal, particularly a human the symptoms of dementia, to slow the progression of dementia, or to reduce in patients with symptoms of dementia the risk of getting worse.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Some of the compounds of the present invention are capable of forming salts with various inorganic and organic acids and bases and such salts are also within the scope of this invention. For example, such conventional non-toxic salts include those derived from inorganic acids such as hydrochloric, phosphoric, and the like; and the salts prepared from organic acids such as lactic, maleic, citric, benzoic, methanesulfonic, trifluoroacetate and the like.

In some embodiments, the present invention provides a compound of formula Ia or Ib or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms. Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975.

The compounds of the invention may be derivatised in various ways. As used herein “derivatives” of the compounds includes salts (e.g. pharmaceutically acceptable salts), any complexes (e.g. inclusion complexes or clathrates with compounds such as cyclodextrins, or coordination complexes with metal ions such as Mn²⁺ and Zn²⁺), esters such as in vivo hydrolysable esters, free acids or bases, polymorphic forms of the compounds, solvates (e.g. hydrates), prodrugs or lipids, coupling partners and protecting groups. By “prodrugs” is meant for example any compound that is converted in vivo into a biologically active compound.

Salts of the compounds of the invention are preferably physiologically well tolerated and non toxic. Many examples of salts are known to those skilled in the art. All such salts are within the scope of this invention, and references to compounds include the salt forms of the compounds.

Compounds having acidic groups, such as carboxylate, phosphates or sulfates, can form salts with alkaline or alkaline earth metals such as Na, K, Mg and Ca, and with organic amines such as triethylamine and Tris (2-hydroxyethyl)amine. Salts can be formed between compounds with basic groups, e.g. amines, with inorganic acids such as hydrochloric acid, phosphoric acid or sulfuric acid, or organic acids such as acetic acid, citric acid, benzoic acid, fumaric acid, or tartaric acid. Compounds having both acidic and basic groups can form internal salts.

Acid addition salts may be formed with a wide variety of acids, both inorganic and organic. Examples of acid addition salts include salts formed with hydrochloric, hydriodic, phosphoric, nitric, sulphuric, citric, lactic, succinic, maleic, malic, isethionic, famaric, benzenesulphonic, toluenesulphonic, methanesulphonic, ethanesulphonic, naphthalenesulphonic, valeric, acetic, propanoic, butanoic, malonic, glucuronic and lactobionic acids.

If the compound is anionic, or has a functional group which may be anionic (e.g., COOH may be COO), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds contain an amine function, these may form quaternary ammonium salts, for example by reaction with an alkylating agent according to methods well known to the skilled person. Such quaternary ammonium compounds are within the scope of the invention.

Compounds containing an amine function may also form N-oxides. A reference herein to a compound that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Esters can be formed between hydroxyl or carboxylic acid groups present in the compound and an appropriate carboxylic acid or alcohol reaction partner, using techniques well known in the art. Examples of esters are compounds containing the group C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, C(═O)OCH₃, C(═O)OCH₂CH₃, C(═O)OC(CH₃)₃, and —C(═O)OPh. Examples of acyloxy (reverse ester) groups are represented by OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, OC(═O)CH₃ (acetoxy), OC(═O)CH₂CH₃, OC(═O)C(CH₃)₃, OC(═O)Ph, and OC(═O)CH₂Ph.

Derivatives which are prodrugs of the compounds are convertible in vivo or in vitro into one of the parent compounds. Typically, at least one of the biological activities of compound will be reduced in the prodrug form of the compound, and can be activated by conversion of the prodrug to release the compound or a metabolite of it. Some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is: C₁₋₇alkyl (e.g., Me, Et, -nPr, -iPr, -nBu, -sBu, -iBu, tBu); C₁₋₇aminoalkyl (e.g., aminoethyl; 2-(N,N-diethylamino)ethyl; 2(4morpholino)ethyl); and acyloxy-C₁₋₇alkyl (e.g., acyloxymethyl; acyloxyethyl; pivaloyloxymethyl; acetoxymethyl; 1acetoxyethyl; 1-(1-methoxy-1-methyl)ethyl-carbonyloxyethyl; 1-(benzoyloxy)ethyl; isopropoxy-carbonyloxymethyl; 1isopropoxy-carbonyloxyethyl; cyclohexyl-carbonyloxymethyl; 1cyclohexyl-carbonyloxyethyl; cyclohexyloxy-carbonyloxymethyl; 1-cyclohexyloxy-carbonyloxyethyl; (4-tetrahydropyranyloxy)carbonyloxymethyl; 1-(4-tetrahydropyranyloxy)carbonyloxyethyl; (4-tetrahydropyranyl)carbonyloxymethyl; and 1(4tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Other derivatives include coupling partners of the compounds in which the compounds is linked to a coupling partner, e.g. by being chemically coupled to the compound or physically associated with it. Examples of coupling partners include a label or reporter molecule, a supporting substrate, a carrier or transport molecule, an effector, a drug, an antibody or an inhibitor. Coupling partners can be covalently linked to compounds of the invention via an appropriate functional group on the compound such as a hydroxyl group, a carboxyl group or an amino group. Other derivatives include formulating the compounds with liposomes.

Where the compounds contain chiral centres, all individual optical forms such as enantiomers, epimers and diastereoisomers, as well as racemic mixtures of the compounds are within the scope of the invention.

Compounds may exist in a number of different geometric isomeric, and tautomeric forms and references to compounds include all such forms. For the avoidance of doubt, where a compound can exist in one of several geometric isomeric or tautomeric forms and only one is specifically described or shown, all others are nevertheless embraced by the scope of this invention.

The quantity of the compound to be administered will vary for the patient being treated and will vary from about 100 ng/kg of body weight to 100 mg/kg of body weight per day and preferably will be from 10 pg/kg to 10 mg/kg per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention.

Compounds of the present invention have been shown to inhibit beta secretase (including BACE) activity in vitro. Inhibitors of beta secretase have been shown to be useful in blocking formation or aggregation of Aβ peptide and therefore have a beneficial effects in treatment of Alzheimer's Disease and other neurodegenerative diseases associated with elevated levels and/or deposition of Aβ peptide. Therefore it is believed that the compounds of the present invention may be used for the treatment of Alzheimer disease and disease associated with dementia Hence compounds of the present invention and their salts are expected to be active against age-related diseases such as Alzheimer, as well as other Aβ related pathologies such as Downs syndrome and b-amyloid angiopathy. It is expected that the compounds of the present invention would most likely be used in combination with a broad range of cognition deficit enhancement agents but could also be used as a single agent.

Generally, the compounds of the present invention have been identified in one or both assays described below as having an IC₅₀ value of 100 micromolar or less.

### IGEN Assay

Enzyme is diluted 1:30 in 40 mM MES pH 5.0. Stock substrate is diluted to 12 W in 40 mM MES pH 5.0. PALMEB solution is added to the substrate solution (1:100 dilution). DMSO stock solutions of compounds or DMSO alone are diluted to the desired concentration in 40 mM MES pH 5.0. The assay is done in a 96 well PCR plate from Nunc. Compound in DMSO (3 μL) is added to the plate then enzyme is added (27 μL) and pre-incubated with compound for 5 minutes. Then the reaction is started with substrate (30 μL). The final dilution of enzyme is 1:60; the final concentration of substrate is 6 μM (Km is 150 μM). After a 20 minute reaction at room temperature, the reaction is stopped by removing 10 μl of the reaction mix and diluting it 1:25 in 0.20M Tris pH 8.0. The compounds are added to the plate by hand then all the rest of the liquid handling is done on the CyBi-well instrument.

All antibodies and the streptavidin coated beads are diluted into PBS containing 0.5% BSA and 0.5% Tween20. The product is quantified by adding 50 μL of a 1:5000 dilution of the neoepitope antibody to 50 μL of the 1:25 dilution of the reaction mix. Then, 100 μL of PBS (0.5% BSA, 0.5% Tween20) containing 0.2 mg/ml IGEN beads and a 1:5000 dilution of ruthinylated goat anti-rabbit (Ru-Gar) antibody is added. The final dilution of neoepitope antibody is 1:20,000, the final dilution of Ru-GAR is 1:10,000 and the final concentration of beads is 0.1 mg/ml. The mixture is read on the IGEN instrument with the CindyAB40 program after a 2-hour incubation at room temperature. Addition of DMSO alone is used to define the 100% activity. 20 μM control inhibitor is used to define 0% of control activity and 100 nM inhibitor defines 50% control of control activity in single-poke assays. Control inhibitor is also used in dose response assays with an IC50 of 100 nM.

### Fluorescent Assay

Enzyme is diluted 1:30 in 40 mM MES pH 5.0. Stock substrate is diluted to 30 μM in 40 mM MES pH 5.0. PALMEB solution is added to the substrate solution (1:100 dilution). Enzyme and substrate stock solutions are kept on ice until the placed in the stock plates. The Platemate-plus instrument is used to do all liquid handling. Enzyme (9 mL) is added to the plate then 1 μL of compound in DMSO is added and pre-incubated for 5 minutes. When a dose response curve is being tested for a compound, the dilutions are done in neat DMSO and the DMSO stocks are added as described above. Substrate (10 μL) is added and the reaction proceeds in the dark for 1 hour at room temperature. The assay is done in a Corning 384 well round bottom, low volume, non-binding surface (Corning #3676). The final dilution of enzyme is 1:60; the final concentration of substrate is 15 μM (Km of 25 μM). The fluorescence of the product is measured on a Victor II plate reader with an excitation wavelength of 360 nm and an emission wavelength of 485 nm using the protocol labeled Edans peptide. The DMSO control defines the 100% activity level and 0% activity is defined by using 50 μM of the control inhibitor, which completely blocks enzyme function. The control inhibitor is also used in dose response assays and has an IC50 of 95 nM.

### Beta-Secretase Whole Cell Assay

### Generation of BEK-Fc33-1:

The cDNA encoding full length BACE was fused in frame with a three amino acid linker (Ala-Val-Thr) to the Fc portion of the human IgG1 starting at amino acid 104. The BACE-Fc construct was then cloned into a GFP/pGEN-IRES-neoK vector (a proprietary vector of AstraZeneca) for protein expression in mammalian cells. The expression vector was stably transfected into HEK-293 cells using a calcium phosphate method. Colonies were selected with 250 μg/mL of G-418. Limited dilution cloning was performed to generate homogeneous cell lines. Clones were characterized by levels of APP expression and Aβ secreted in the conditioned media using an ELISA assay developed in-house. Aβ secretion of BACE/Fc clone Fc33-1 was moderate.

### Cell Culture:

HEK293 cells stably expressing human BACE (HEK-Fc33) were grown at 37° C. in DMEM containing 10% heat-inhibited FBS, 0.5 mg/mL antibiotic-antimycotic solution, and 0.05 mg/mL of the selection antibiotic G-418.

### Aβ40 Release Assay:

Cells were harvested when between 80 to 90% confluent. 100 μL of cells at a cell density of 1.5 million/mL were added to a white 96-well cell culture plate with clear flat bottom (Costar 3610), or a clear, flat bottom 96-well cell culture plate (Costar 3595), containing 100 μL of inhibitor in cell culture medium with DMSO at a final concentration of 1%. After the plate was incubated at 37° C. for 24 h, 100 μL cell medium was transferred to a round bottom 96-well plate (Costar 3365) to quantify Aβ40 levels. The cell culture plates were saved for ATP assay as described in ATP assay below. To each well of the round bottom plate, 50 μL of detection solution containing 0.2 μg/mL of the RαAβ40 antibody and 0.25 μg/mL of a biotinylated 4G8 antibody (prepared in DPBS with 0.5% BSA and 0.5% Tween-20) was added and incubated at 4° C. for at least 7 h. Then a 50 μL solution (prepared in the same buffer as above) containing 0.062 μg/mL of a ruthenylated goat anti-rabbit antibody and 0.125 mg/mL of streptavidin coated Dynabeads was added per well. The plate was shaken at 22° C. on a plate shaker for 1 h, and then the plates were then measured for ECL counts in an IGEN M8 Analyzer. Aβ standard curves were obtained with 2-fold serial dilution of an Aβ stock solution of known concentration in the same cell culture medium used in cell-based assays.

### ATP Assay:

As indicated above, after transferring 100 μL medium from cell culture plates for A040 detection, the plates, which still contained cells, were saved for cytotoxicity assays by using the assay kit (ViaLight™ Plus) from Cambrex BioScience that measures total cellular ATP. Briefly, to each well of the plates, 50 μL cell lysis reagent was added. The plates were incubated at room temperature for 10 min. Two min following addition of 100 μL reconstituted ViaLight™ Plus reagent for ATP measurement, the luminescence of each well was measured in an LJL plate reader or Wallac Topcount.

### BACE Biacore Protocol

### Sensor Chip Preparation:

BACE was assayed on a Biacore3000 instrument by attaching either a peptidic transition state isostere (TSI) or a scrambled version of the peptidic TSI to the surface of a Biacore CM5 sensor chip. The surface of a CM5 sensor chip has 4 distinct channels that can be used to couple the peptides. The scrambled peptide KFES-statine-ETIAEVENV was coupled to channel 1 and the TSI inhibitor KTEEISEVN-statine-VAEF was couple to channel 2 of the same chip. The two peptides were dissolved at 0.2 mg/ml in 20 mM Na Acetate pH 4.5, and then the solutions were centrifuged at 14K rpm to remove any particulates. Carboxyl groups on the dextran layer were activated by injecting a one to one mixture of 0.5M N-ethyl-N′ (3-dimethylaminopropyl)-carbodiimide (EDC) and 0.5M N-hydroxysuccinimide (NHS) at 5 μL/minute for 7 minutes. Then the stock solution of the control peptide was injected in channel 1 for 7 minutes at 5 μL/min., and then the remaining activated carboxyl groups were blocked by injecting IM ethanolamine for 7 minutes at 5 μL/minute.

### Assay Protocol:

The BACE Biacore assay was done by diluting BACE to 0.5 μM in Na Acetate buffer at pH 4.5 (running buffer minus DMSO). The diluted BACE was mixed with DMSO or compound diluted in DMSO at a final concentration of 5% DMSO. The BACE/inhibitor mixture was incubated for 1 hour at 4° C. then injected over channel 1 and 2 of the CM5 Biacore chip at a rate of 20 μL/minute. As BACE bound to the chip the signal was measured in response units (RU). BACE binding to the TSI inhibitor on channel 2 gave a certain signal. The presence of a BACE inhibitor reduced the signal by binding to BACE and inhibiting the interaction with the peptidic TSI on the chip. Any binding to channel 1 was non-specific and was subtracted from the channel 2 responses. The DMSO control was defined as 100% and the effect of the compound was reported as percent inhibition of the DMSO control.

hERG Assay

### Cell Culture

The hERG-expressing Chinese hamster ovary KI (CHO) cells described by (Persson, Carlsson, Duker, & Jacobson, 2005) were grown to semi-confluence at 37° C. in a humidified environment (5% CO₂) in F-12 Ham medium containing L-glutamine, 10% foetal calf serum (FCS) and 0.6 mg/ml hygromycin (all Sigma-Aldrich). Prior to use, the monolayer was washed using a pre-warmed (37° C.) 3 ml aliquot of Versene 1:5,000 (Invitrogen). After aspiration of this solution the flask was incubated at 37° C. in an incubator with a further 2 ml of Versene 1:5,000 for a period of 6 minutes. Cells were then detached from the bottom of the flask by gentle tapping and 10 ml of Dulbecco's Phosphate-Buffered Saline containing calcium (0.9 mM) and magnesium (0.5 mM) (PBS; Invitrogen) was then added to the flask and aspirated into a 15 ml centrifuge tube prior to centrifugation (50 g, for 4 mins). The resulting supernatant was discarded and the pellet gently re-suspended in 3 ml of PBS. A 0.5 ml aliquot of cell suspension was removed and the number of viable cells (based on trypan blue exclusion) was determined in an automated reader (Cedex; Innovatis) so that the cell re-suspension volume could be adjusted with PBS to give the desired final cell concentration. It is the cell concentration at this point in the assay that is quoted when referring to this parameter. CHO-Kv1.5 cells, which were used to adjust the voltage offset on IonWorks™ HT, were maintained and prepared for use in the same way.

### Electrophysiology

The principles and operation of this device have been described by (Schroeder, Neagle, Trezise, & Worley, 2003). Briefly, the technology is based on a 384-well plate (PatchPlate™) in which a recording is attempted in each well by using suction to position and hold a cell on a small hole separating two isolated fluid chambers. Once sealing has taken place, the solution on the underside of the PatchPlate™ is changed to one containing amphotericin B. This permeablises the patch of cell membrane covering the hole in each well and, in effect, allows a perforated, whole-cell patch clamp recording to be made.

A β-test IonWorks™ HT from Essen Instrument was used. There is no capability to warm solutions in this device hence it was operated at room temperature (˜21° C.), as follows. The reservoir in the “Buffer” position was loaded with 4 ml of PBS and that in the “Cells” position with the CHO-HERG cell suspension described above. A 96-well plate (V-bottom, Greiner Bio-one) containing the compounds to be tested (at 3-fold above their final test concentration) was placed in the “Plate 1” position and a Patchplate™ was clamped into the PatchPlate™ station. Each compound plate was laid-out in 12 columns to enable ten, 8-point concentration-effect curves to be constructed; the remaining two columns on the plate were taken up with vehicle (final concentration 0.33% DMSO), to define the assay baseline, and a supra-maximal blocking concentration of cisapride (final concentration 10 μM) to define the 100% inhibition level. The fluidics-head (F-Head) of IonWorks™ HT then added 3.5 μl of PBS to each well of the PatchPlate™ and its underside was perfused with “internal” solution that had the following composition (in mM): K-Gluconate 100, KCl 40, MgCl₂ 3.2, EGTA 3 and HEPES 5 (all Sigma-Aldrich; pH 7.25-7.30 using 10 M KOH). After priming and de-bubbling, the electronics-head (E-head) then moved round the PatchPlate™ performing a hole test (i.e. applying a voltage pulse to determine whether the hole in each well was open). The F-head then dispensed 3.5 μL of the cell suspension described above into each well of the PatchPlate™ and the cells were given 200 seconds to reach and seal to the hole in each well. Following this, the E-head moved round the PatchPlate™ to determine the seal resistance obtained in each well. Next, the solution on the underside of the PatchPlate™ was changed to “access” solution that had the following composition (in mM): KCl 140, EGTA 1, MgCl₂ 1 and HEPES 20 (pH 7.25-7.30 using 10 M KOH) plus 100 μg/ml of amphotericin B (Sigma-Aldrich). After allowing 9 minutes for patch perforation to take place, the E-head moved round the PatchPlate™ 48 wells at a time to obtain pre-compound hERG current measurements. The F-head then added 3.5 μl of solution from each well of the compound plate to 4 wells on the PatchPlate™ (the final DMSO concentration was 0.33% in every well). This was achieved by moving from the most dilute to the most concentrated well of the compound plate to minimise the impact of any compound carry-over. After approximately 3.5 mins incubation, the E-head then moved around all 384-wells of the PatchPlate™ to obtain post-compound hERG current measurements. In this way, non-cumulative concentration-effect curves could be produced where, providing the acceptance criteria were achieved in a sufficient percentage of wells (see below), the effect of each concentration of test compound was based on recording from between 1 and 4 cells.

The pre- and post-compound hERG current was evoked by a single voltage pulse consisting of a 20 s period holding at −70 mV, a 160 ms step to −60 mV (to obtain an estimate of leak), a 100 ms step back to −70 mV, a 1 s step to +40 mV, a 2 s step to −30 mV and finally a 500 ms step to −70 mV. In between the pre- and post-compound voltage pulses there was no clamping of the membrane potential. Currents were leak-subtracted based on the estimate of current evoked during the +10 mV step at the start of the voltage pulse protocol. Any voltage offsets in IonWorks™ HT were adjusted in one of two ways. When determining compound potency, a depolarising voltage ramp was applied to CHO-Kv1.5 cells and the voltage noted at which there was an inflection point in the current trace (i.e. the point at which channel activation was seen with a ramp protocol). The voltage at which this occurred had previously been determined using the same voltage command in conventional electrophysiology and found to be −15 mV (data not shown); thus an offset potential could be entered into the IonWorks™ HT software using this value as a reference point. When determining the basic electrophysiological properties of hERG, any offset was adjusted by determining the hERG tail current reversal potential in IonWorks™ HT, comparing it with that found in conventional electrophysiology (−82 mV; see FIG. 1c) and then making the necessary offset adjustment in the IonWorks™ HT software. The current signal was sampled at 2.5 kHz.

Pre- and post-scan hERG current magnitude was measured automatically from the leak subtracted traces by the IonWorks™ HT software by taking a 40 ms average of the current during the initial holding period at −70 mV (baseline current) and subtracting this from the peak of the tail current response. The acceptance criteria for the currents evoked in each well were: pre-scan seal resistance >60 MΩ, pre-scan HERG tail current amplitude >150 pA; post-scan seal resistance >60 MΩ. The degree of inhibition of the hERG current was assessed by dividing the post-scan hERG current by the respective pre-scan hERG current for each well.

### Methods of Preparation

The compounds of the present invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those skilled in the art. Such methods include, but are not limited to, those described below. All references cited herein are hereby incorporated in their entirety by reference.

The novel compounds of this invention may be prepared using the reactions and techniques described herein. The reactions are performed in solvents appropriate to the reagents and materials employed and are suitable for the transformations being effected. Also, in the description of the synthetic methods described below, it is to be understood that all proposed reaction conditions, including choice of solvent, reaction atmosphere, reaction temperature, duration of the experiment and workup procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents, which are not compatible with the reaction conditions, will be readily apparent to one skilled in the art and alternate methods must then be used.

The starting materials for the examples contained herein are either commercially available or are readily prepared by standard methods from known materials. For example the following reactions are illustrations but not limitations of the preparation of some of the starting materials and examples used herein.

General procedures for making the compounds of the invention is as follows:

The invention will now be illustrated by the following nonlimiting examples.

### Normal Phase Chromatography Conditions:

Flash chromatography employed as a method for purification for selected compounds and intermediates. Isco CombiFlash Sq 16× instrument: pre-packaged disposable RediSep SiO₂ stationary phase columns (4, 12, 40, 120 gram sizes) with gradient elution at 5-125 mL/min of selected bi-solvent mixture, UV detection (190-760 nm range) or timed collection, 0.1 mm flow cell path length.

### Microwave Heating Instrumentation:

A Personal Chemistry Smith Synthesizer unit (monomodal, 2.45 GHz, 300 W max) was utilized for microwave heating of reactions.

### LC-MS HPLC Conditions:

Column: Agilent Zorbax SB-C8 2 mm ID×50 mm Flow: 1.4 mL/min

Gradient: 95% A to 90% B over 3 min hold 1 minute ramp down to 95% A over 1 minute and hold 1 minute. Where A=2% acetonitrile in water with 0.1% formic acid and B=2% water in acetonitrile with 0.1% formic acid. UV-DAD 210-400 nm.

### Abbreviations:

APCI: atmospheric pressure chemical ionization; DME: 1,2 dimethoxyethane; DMF: HPLC: high pressure liquid chromatography; NMR: nuclear magnetic resonance; GMF: Glass Microfiber Filter.

### General Experimental Detail:

For mass spectral data, results are reported in units of m/z for the parent ion (M+1) unless otherwise indicated. In cases where isotopic splitting (for example, with compounds containing bromine) results in multiple peaks, only the major peak in the cluster is indicated. NMR data are reported for key resonances, were recorded in the indicated deuterated solvent, and chemical shifts are reported in parts per million relative to tetramethyl silane.

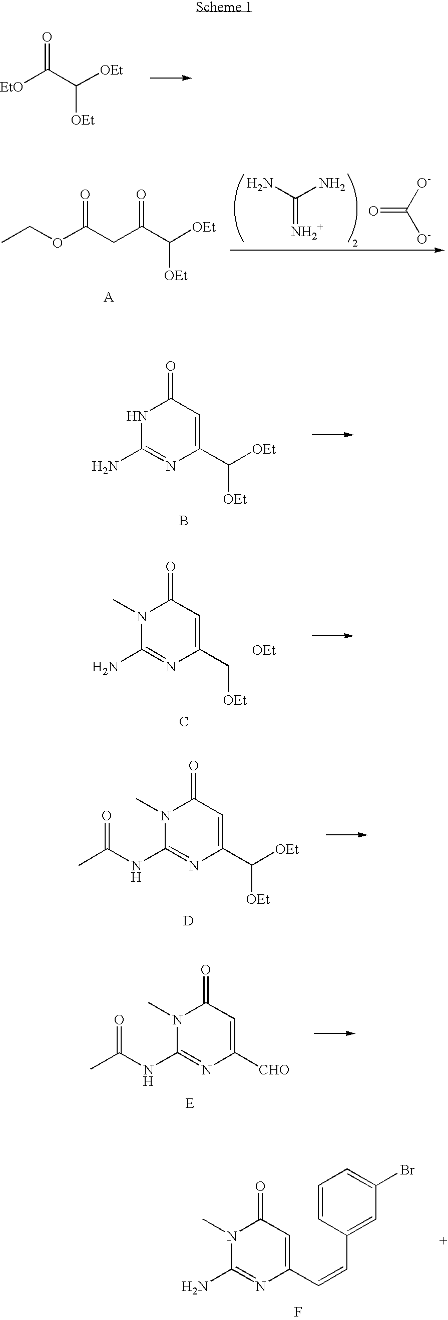

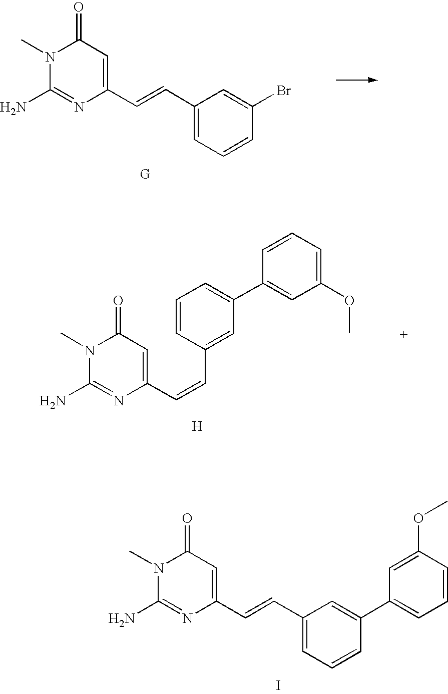

### EXAMPLE 1

### 2-Amino-6-[(Z)-2-(3′-methoxybiphenyl-3-yl)vinyl]-3-methylpyrimidin-4(3H)-one (Scheme 1, H)

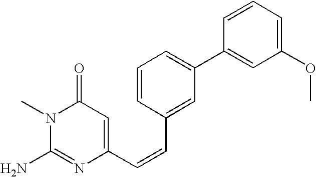

A thick-walled glass vial was charged with a stir bar, a mixture of the geometric isomers 2-amino-6-[(Z)-2-(3-bromophenyl)vinyl]-3-methylpyrimidin-4(3H)-one (Scheme 1, F) and 2-amino-6-[(E)-2-(3-bromophenyl)vinyl]-3-methylpyrimidin-4(3H)-one (Scheme 1, G) (approximately 1:1, 18 mg, 0.06 mmol), 3-methoxyphenylboronic acid (9.2 mg, 0.07 mmol), dichlorobis(triphenylphosphine)-palladium (II) (approximately 1 mg, 0.001 mmol), Cs₂CO₃ (39 mg, 0.12 mmol) and DME/H₂O/ethanol (7:3:2; approximately 1.2 mL). The reaction vial was sealed and subject to microwave radiation for 10 min at 150° C. The resultant black slurry was filtered through diatomaceous earth/MgSO₄ pad in a 0.7 μm GMF filter, washing liberally with ethanol and methylene chloride (3×1 mL each) then concentrated in vacuo. The resultant residue was subject to flash chromatography (SiO₂—4 g; gradient elution: 30-100% ethyl acetate/hexane over 24 min at 20 mL/min) to provide the 2-amino-6-[(Z)-2-(3′-methoxybiphenyl-3-yl)vinyl]-3-methylpyrimidin-4(3H)-one as a clear film upon concentration (2.7 mg, 28% based on 1:1 starting mixture stoichiometry). ¹H NMR (300 MHz, CDCl₃) δ 3.39 (s, 3H), 3.85 (s, 3H), 4.97 (br s, 2H), 5.93 (s, 1H), 6.25 (d, J=12.5 Hz, 1H), 6.80 (d, J=12.5 Hz, 1H), 6.88 (dd, J=8.1, 2.2 Hz, 1H), 7.05 (t, J=1.9 Hz, 1H), 7.12 (d, J=7.8 Hz, 1H), 7.30-7.35 (m, 3H), 7.45 (dd, J=1.6, 3.5 Hz, 1H), 7.61 (s, 1H); m/z (ES+) M+1=334.1; HPLC t_{R}=1.82 min.

The requisite 2-amino-6-[(Z)-2-(3-bromophenyl)vinyl]-3-methylpyrimidin-4(3H)-one (Scheme 1, F) and 2-amino-6-[(E)-2-(3-bromophenyl)vinyl]-3-methylpyrimidin-4(3H)-one (Scheme 1, G) were prepared as follows.

### Ethyl 4,4-diethoxy-3-oxobutahoate (Scheme 1, A)

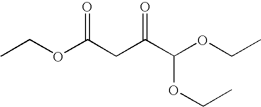

To a stirring suspension of sodium hydride (5.12 g, 213 mmol) in anhydrous tetrahydrofuran (250 mL) at 50° C. under an argon atmosphere was added dropwise a mixture of ethyl acetate (15.3 mL, 156 mmol) and ethyl diethoxyacetate (25 mL, 142 mmol) over 30 min. Upon complete addition the reaction was brought to reflux for 4 hours before allowing to cool to ambient temperature with continued stirring for an additional 16 hours. The mixture was concentrated to approximately one-third of the original volume by rotary evaporation before quenching by the rapid addition to aqueous acetic acid (approximately 145 mL, 15% v/v) at 0° C. followed by extraction with diethyl ether (4×100 mL). The combined organic extracts were washed sequentially with water (1×20 mL), a saturated sodium carbonate aqueous solution (3×50 mL), water (2×20 mL), brine (1×30 mL), then dried over MgSO₄ and concentrated in vacuo to afford the desired ethyl 4,4-diethoxy-3-oxobutanoate (Scheme 1, A) as a clear yellow oil as a 4:1 mixture of keto-enol tautomers (25.05 g, 81%). ¹H NMR (300 MHz, CDCl₃) δ 1.22-1.30 (m, 9H), 3.53-3.76 (m, 5.6H), 4.19 (q, J=7.6 Hz, 2H), 4.67 (s, 0.8H), 4.92 (s, 0.2H), 5.45 (s, 0.2H), 11.88 (s, 0.2H).

### 2-Amino-6-(diethoxymethyl)pyrimidin-4(3H)-one (Scheme 1, B)

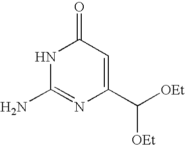

To a stirring solution of ethyl 4,4-diethoxy-3-oxobutanoate (Scheme 1, A) (approximately 25 g, 114 mmol) in ethanol (600 mL) was added guanidine carbonate (10.42 g, 57.8 mmol), washing in with additional ethanol (200 mL) before the reaction was heated under reflux for 18 h. Upon cooling the resultant white crystals were collected by filtration, washing with cold ethanol. Subsequent concentrations of the filtrate to reduced volumes by rotary evaporation and cooling afforded additional crops of white crystals, which were collected by filtration and washed with cold ethanol. The crops were dried under high vacuum at ambient temperature over night to afford the title compound as a white solid (15.5 g, 64%). ¹H NMR (300 MHz, DMSO-d₆) δ 1.13 (t, J=7.1 Hz, 6H), 3.45-3.58 (m, 4H), 4.90 (s, 1H), 5.62 (s, 1H), 6.57 (br s, 2H), 10.73 (br s, 1H); m/z (ES+) M+1=214.1; HPLC t_{R}=0.70 min.

### 2-Amino-6-(diethoxymethyl)-3-methylpyrimidin-4(3H)-one (Scheme 1, C)

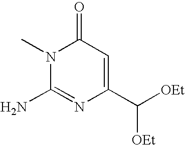

To a suspension of 2-amino-6-(diethoxymethyl)pyrimidin-4(3H)-one (Scheme 1, B) (1.02 g, 4.8 mmol) in absolute ethanol (30 mL) was added solid potassium hydroxide (484 mg, 8.6 mmol), which was stirred until a homogeneous solution was achieved. Iodomethane (1.08 mL, 17.3 mmol) was added in one portion and the reaction heated in a sealed tube to 78° C. for 17 h. Upon completion, the mixture was concentrated in vacuo to a pale golden residue and subject to flash chromatography (SiO₂, 40 g; gradient elution: 2-5% methanol/CH₂Cl₂ over 25 min at 60 mL/min) to give rise to the 2-amino-6-(diethoxymethyl)-3-methylpyrimidin-4(3H)-one (Scheme 1, C) as a pale yellow solid (833 mg, 76%). ¹H NMR (300 MHz, CDCl₃) δ 1.25 (t, J=7.1 Hz, 6H), 2.01 (br s, 2H), 3.44 (s, 3H), 3.55-3.68 (m, 4H), 5.07 (s, 1H), 6.18 (s, 1H); m/z (ES+) M+1=228.3; HPLC t_{R}=0.87 min.

### N-[4-(Diethoxymethyl)-1-methyl-6-oxo-1,6-dihydropyrimidin-2-yl]acetamide (Scheme 1, D)

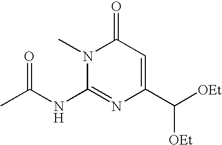

To a stirring dichloromethane (40 mL) solution of 2-amino-6-(diethoxymethyl)-3-methylpyrimidin-4(3H)-one (Scheme 1, C) (1.33 g, 5.83 mmol) was added acetic anhydride (2.2 mL, 23.2 mmol) and N,N-dimethylpyridin-4-amine (71 mg, 0.58 mmol) at ambient temperature. After 5 h the reaction was washed with water (1×20 mL), dried over MgSO₄ and concentrated in vacuo. The tacky yellow residue was subject to flash chromatography (SiO₂, 40 g; gradient elution: 10% ethyl acetate/hexane for 2 min then 10-100% ethyl acetate/hexane over 20 min at 60 mL/min) to yield the title compound N-[4-(diethoxymethyl)-1-methyl-6-oxo-1,6-dihydropyrimidin-2-yl]acetamide (Scheme 1, D) as an off-white solid (1.1 g, 70%). ¹H NMR (300 MHz, CDCl₃) δ 1.29 (t, J=7.1 Hz, 6H), 2.24 (s, 3H), 3.46 (s, 3H), 3.65 (m, J=7.1 Hz, 4H), 5.28 (s, 1H), 6.05 (s, 1H), 13.76 (br s, 1H); m/z (ES+) M+1=270.3; HPLC t_{R}=1.30 min.

### N-(4-Formyl-1-methyl-6-oxo-1,6-dihydropyrimidin-2-yl)acetamide (Scheme 1, E)

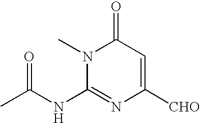

A glass vial was charged with a stir bar, N-[4-(diethoxymethyl)-1-methyl-6-oxo-1,6-dihydropyrimidin-2-yl]acetamide (Scheme 1, D) (500 mg, 1.86 mmol) and formic acid (4 mL). The vial was immersed in a pre-heated oil bath at 100° C. for 20 min before being concentrated down by rotary evaporation. The residue was taken up in hot ethyl acetate, cooled, filtered, and washed liberally with diethyl ether. The filtrate was then concentrated and subject to treatment with diethyl ether, followed by filtration. Concentration of the mother liquor and drying in vacuo afforded the title compound as a white solid (148 mg, 41%). ¹H NMR (300 MHz, DMSO-d₆) δ 2.17 (s, 3H), 3.35 (s, 3H), 6.81 (s, 1H), 9.75 (s, 1H), 10.89 (s, 1H); HPLC t_{R}=0.91 min.

### 2-Amino-6-[(Z)-2-(3-bromophenyl)vinyl]-3-methylpyrimidin-4(3H)-one (Scheme 1, F)

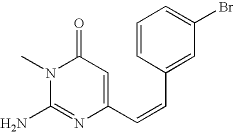

To a stirring suspension of N-(4-formyl-1-methyl-6-oxo-1,6-dihydropyrimidin-2-yl)acetamide (Scheme 1, E) (331 mg, 1.70 mmol) and (3-bromo-benzyl)-triphenylphosphonium chloride (793 mg, 1.70 mmol) (Beak, et al., Tetrahedron, 1994, 50, 5999) in anhydrous tetrahydrofuran (10 mL) at 0° C. under an argon atmosphere was added rapidly via syringe potassium tert-butoxide in 2-methyl-2-propanol (1.0 M, 3.56 mL, 3.56 mmol). Allowed to warm to ambient temperature and left to stir for 22 h. The reaction was quenched by addition of aqueous HCl (0.5N) adjusting to pH˜5, dilute with water, and extracted with methylene chloride (3×20 mL). The combined organics were dried over MgSO₄ and concentrated in vacuo to a residue that was subject to flash chromatography (SiO₂—12 g; gradient elution: 35-100% ethyl acetate/hexane over 20 min at 40 mL/min). Trituration of the resultant film with methanol afforded the 2-amino-6-[(Z)-2-(3-bromophenyl)vinyl]-3-methylpyrimidin-4(3H)-one (Scheme 1, F) as an off-white solid (21 mg, 4%). ¹H NMR (300 MHz, DMSO-d₆) δ 2.49 (s, 3H), 3.90 (s, 2H), 4.74 (s, 1H), 5.41 (d, J=12.5 Hz, 1H), 5.86 (d, J=12.5 Hz, 1H), 6.31 (app t, J=7.8 Hz, 1H), 6.41 (d, J=7.8 Hz, 1H), 6.50 (d, J=7.8 Hz, 1H), 6.62 (s, 1H); m/z (ES+) M+1=306.0; HPLC t_{R}=1.53 min.

### 2-Amino-6-[(E)-2-(3-bromophenyl)vinyl]-3-methylpyrimidin-4(3H)-one (Scheme 1, G)

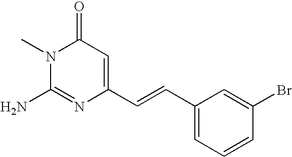

This material was also obtained from the above reaction used to afford 2-amino-6-[(Z)-2-(3-bromophenyl)vinyl]-3-methylpyrimidin-4(3H)-one (Scheme 1, F). The titled material was obtained as a white solid (21 mg, 4%). ¹H NMR (300 MHz, DMSO-d₆) δ 2.52 (s, 3H), 3.90 (s, 2H), 5.01 (s, 1H), 6.00 (d, J=15.7 Hz, 1H), 6.40 (app t, J=7.8 Hz, 1H), 6.57 (d, J=7.8 Hz, 1H), 6.65 (d, J=6.6 Hz, 1H), 6.69 (d, J=15.7 Hz, 1H), 6.85 (s, 1H); m/z (ES+) M+1=306.0; HPLC t_{R}=1.71 min.

### EXAMPLE 2

### 2-Amino-6-[(E)-2-(3′-methoxybiphenyl-3-yl)vinyl]-3-methylpyrimidin-4(3H)-one (Scheme 1, I)

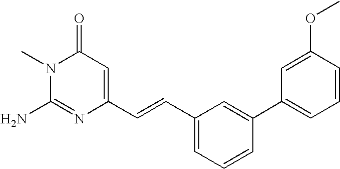

was obtained as an off-white solid from the preparation of the compound of Example 1 (5.6 mg, 57%-based on 1:1 starting mixture stoichiometry). ¹H NMR (300 MHz, CDCl₃) [symbol] 3.45 (s, 3H), 3.88 (s, 3H), 4.96 (br s, 2H), 5.99 (s, 1H), 6.82 (d, J=15.7 Hz, 1H), 6.92 (dd, J=8.1, 1.9 Hz, 1H), 7.13 (t, J=2.0 Hz, 1H), 7.19 (d, J=7.7 Hz, 1H), 7.37 (t, J=8.0 Hz, 1H), 7.44 (d, J=7.5 Hz, 1H), 7.51-7.54 (m, 2H), 7.67 (d, J=15.7 Hz, 1H), 7.73 (s, 1H); m/z (ES+) M+1=334.1; HPLC t_{R}=1.99 min.

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, and the like) cited in the present application is incorporated herein by reference in its entirety.

1. A compound of formula Ia or formula Ib: 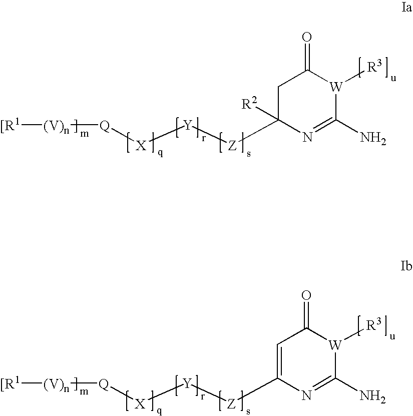
or a pharmaceutically acceptable salt, tautomer or in vivo-hydrolysable precursor thereof, wherein:
W is C or N;
Q is selected from C₃₋₁₂cycloalkyl, C₃₋₁₂cycloalkenyl, C₆₋₁₄aryl, or C₅₋₁₅heterocyclyl; each R¹ is, independently, selected from H, halogen, C₂₋₆alkenyl, C₁₋₆alkyl, C₃₋₁₂cycloalkyl, C₆₋₁₀aryl, C₁₋₁₆alkyl-C₆₋₁₀aryl, or C₅₋₁₅heterocyclyl wherein the C₁₋₁₆alkyl, the C₃₋₁₂cycloalkyl, the C₆₋₁₀aryl, the C₁₋₆alkyl-C₆₋₁₀aryl, or the C₅₋₁₅heterocyclyl is optionally substituted by 1, 2, or 3 substitutents independently selected from: halogen, CN, NH₂, OH, COOH, OC₁₋₆alkyl, CH₂OH, SO₂H, S(═O), C₂₋₆alkenyl, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R³, C(═O)N(C₁₋₆alkyl-R^{a})₂, S(═O)C₁₋₆alkyl-R^{a}, S(═O)NHC₁₋₆alkyl-R³, S(═O)N(C₁₋₆alkyl-R³)₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-R^{a}, N(C₁₋₆alkyl-R^{a})₂, NHC(═O)C₁₋₆alkyl, C₆₋₁₀aryl-R³, OC₆₋₁₀aryl-R^{a}, C(═O)C₆₋₁₀aryl-R^{a}, C(═O)OC₆₋₁₀aryl-R^{a}, C(═O)NHC₆₋₁₀aryl-R^{a}, C(═O)N(C₆₋₁₀aryl-R^{a})₂, S(═O)C₆₋₁₀aryl-R^{a}, S(═O)NHC₆₋₁₀aryl-R^{a}, S(═O)N(C₆₋₁₀aryl-R^{a})₂, SO₂C₆₋₁₀aryl-R^{a}, SO₂NHC₆₋₁₀aryl-R^{a}, SO₂N(C₆₋₁₀aryl-R^{a})₂, NH(C₆₋₁₀aryl)-R^{a}, N(C₆₋₁₀aryl-R³)₂, NC(═O)C₆₋₁₀aryl, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NHC₅₋₆heterocyclyl-R^{a}, C(═O)N(C₅₋₆heterocyclyl-R^{a})₂, S(═O)C₅₋₆heterocyclyl-R^{a}, S(═O)NHC₅₋₆heterocyclyl-R^{a}, S(═O)N(C₅₋₆heterocyclyl-R^{a})₂, SO₂C₅₋₆heterocyclyl-R^{a}, SO₂NHC₅₋₆heterocyclyl-R³, SO₂N(C₅₋₆heterocyclyl-R³)₂, NH(C₅₋₆heterocyclyl-R^{a}), N(C₅₋₆heterocyclyl-R^{a})₂, NHC(═O)C₅₋₆heterocyclyl, SO₂R^{a}, S(═O)R^{a}, N(C₁₋₆alkyl-R^{a})(C₆₋₁₀aryl-R^{a}), N(C₁₋₆alkyl-R^{a})(C₆₋₁₀heteroaryl-R^{a}), N(C₆₋₁₀aryl-R^{a})(C₆₋₁₀heteroaryl-R^{a}), C(═O)(C₁₋₆alkyl-R^{a})(C₆₋₁₀aryl-R^{a}), C(═O)(C₁₋₆alkyl-R^{a})(C₆₋₁₀heteroaryl-R^{a}), C(═O)(C₆₋₁₀aryl-R^{a})(C₆₋₁₀heteroaryl-R^{a}), C(═O)O(C₁₋₆alkyl-R^{a})(C₆₋₁₀aryl-R^{a}), C(═O)O(C₁₋₆alkyl-R^{a})(C₆₋₁₀heteroaryl-R^{a}), C(═O)O(C₆₋₁₀aryl-R^{a})(C₆₋₁₀heteroaryl-R^{a}), S(═O)(C₁₋₆alkyl-R^{a})(C₆₋₁₀aryl-R^{a}), S(═O)(C₁₋₆alkyl-R^{a})(C₆₋₁₀heteroaryl-R^{a}), S(═O)(C₆₋₁₀aryl-R^{a})(C₆₋₁₀heteroaryl-R^{a}), SO₂(C₁₋₆alkyl-R^{a})(C₆₋₁₀aryl-R^{a}), SO₂(C₁₋₆alkyl-R^{a})(C₆₋₁₀heteroaryl-R^{a}), or SO₂(C₆₋₁₀aryl-R^{a})(C₆₋₁₀heteroaryl-R^{a});
each R^{a} is, independently, selected from H, halogen, CN, NH₂, OH, C₁₋₆alkyl, OC₁₋₆alkyl, C(═O)C₁₋₆alkyl, C(═O)OC₁₋₆alkyl, C(═O)NH₂, C(═O)NHC₁₋₆alkyl, C(═O)N(C₁₋₆alkyl)₂, SOC₁₋₆alkyl, SONHC₁₋₆alkyl, SON(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NC(═O)C₁₋₆alkyl, C₅₋₆aryl, OC₅₋₆aryl, C(═O)C₅₋₆aryl, C(═O)OC₅₋₆aryl, C(═O)NH₂, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NC(═O)C₅₋₆aryl, C₅₋₆heterocyclyl, OC₅₋₆heterocyclyl, C(═O)C₅₋₆heterocyclyl, C(═O)OC₅₋₆heterocyclyl, C(═O)NH₂, C(═O)NHC₅₋₆heterocyclyl, C(═O)N(C₅₋₆heterocyclyl)₂, S(═O)C₅₋₆heterocyclyl, S(═O)NHC₅₋₆heterocyclyl, S(═O)N(C₅₋₆heterocyclyl)₂, SO₂NHC₅₋₆heterocyclyl, SO₂N(C₅₋₆heterocyclyl)₂, NH(C₅₋₆heterocyclyl), N(C₅₋₆heterocyclyl)₂, NC(═O)C₅₋₆heterocyclyl, C(═O)NHC₁₋₆alkylC₅₋₆aryl, NR^{b}R^{b}, C(═O)R^{e}, C(═O)NR^{b}R^{b}, OC(═O)NR^{b}R^{b}, S(═O)R^{b}, S(═O)NR^{b}R^{b}, or SO₂NR^{b}R^{b};
each R^{b} is, independently, selected from H, C₁₋₆alkyl, C₅₋₆aryl, or C₅₋₆heterocyclyl;
each V is, independently, selected from NH, O, S, S(═O), SO₂, NHS(═O), NHSO₂, S(═O)NH, SO₂NH, NHC(═O), C(═O)NH, NR^{a}SO₂, NR^{a}S(═O), NR^{a}C(O), C(O)NR^{a}, S(O)₂NR^{a}, S(═O)NR^{a}, OC₁₋₆alkylenyl, C₂₋₆alkenylenyl or C₁₋₆alkylenyl, wherein the OC₁₋₆alkylenyl, C₂₋₆alkenylenyl, and C₁₋₆alkylenyl is optionally substituted by 1, 2, or 3 substitutents independently selected from R^{a};
X and Y are each independently selected from NH, O, S, S(═O), SO₂, NHS(═O), NHSO₂, S(═O)NH, SO₂NH, NHC(═O), C(═O)NH, NR^{a}SO₂, NR^{a}S(═O), NR^{a}C(O), C(O)NR^{a}, S(O)₂NR^{a}, S(═O)NR^{a}, or C₁₋₆alkyl wherein the C₁₋₆alkyl is optionally substituted by 1, 2, or 3 substituents independently selected from R^{a};
Z is 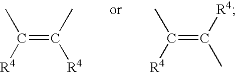
m is 0, 1, 2 or 3;
n, q, r, and u are each, independently, 0 or 1;
s is 1 or 2;
R² is selected from H, halogen, C₁₋₆alkyl, C₃₋₁₂cycloalkyl, C₆₋₁₀aryl, C₁₋₆alkyl-C₆₋₁₀aryl, C₅₋₁₀heterocyclyl, or C₁₋₆alkyl-C₅₋₁₀heterocyclyl wherein the C₁₋₆alkyl, C₃₋₁₂cycloalkyl, C₆₋₁₀aryl, C₁₋₁₆alkyl-C₆₋₁₀aryl, C₅₋₁₀heterocyclyl, and C₁₋₁₆alkyl-C₅₋₁₀heterocyclyl is optionally substituted by 1, 2, or 3 substitutents independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂ S(═O)C₁₋₆alkyl-R^{a}, S(═O)NHC₁₋₆alkyl-R^{a}, S(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-R^{a}, N(C₁₋₆alkyl-R^{a})₂, NHC(═O)C₁₋₆alkyl, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl-R^{a})₂, S(═O)C₅₋₆aryl-R^{a}, S(═O)NHC₅₋₆aryl-R^{a}, S(═O)N(C₅₋₆aryl-R^{a})₂, SO₂C₅₋₆aryl-R^{a}, SO₂NHC₅₋₆aryl-R^{a}, SO₂N(C₅₋₆aryl-R^{a})₂, NH(C₅₋₆aryl)-R^{a}, N(C₅₋₆aryl-R^{a})₂, NHC(═O)C₅₋₆aryl, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl-R³)₂, SO₂C₅₋₆heterocyclyl-R^{a}, SO₂NHC₅₋₆heterocyclyl-R^{a}, SO₂N(C₅₋₆heterocyclyl-R^{a})₂ S(═O)C₅₋₆heterocyclyl-R^{a}, S(═O)NHC₅₋₆heterocyclyl-R^{a}, S(═O)N(C₅₋₆heterocyclyl-R^{a})₂, NH(C₅₋₆heterocyclyl)-R^{a}, N(C₅₋₆heterocyclyl-R^{a})₂, or NHC(═O)C₅₋₆heterocyclyl;
R³ is selected from R¹, C₁₋₆alkylR^{c}, C₁₋₆alkylNR^{c}R^{c}, C₁₋₆alkylOR^{c}, C₁₋₆alkylSR^{c}, C₁₋₆alkylNHC₁₋₆alkylC₅₋₆arylR^{d}, C₁₋₆alkyNHC₆₋₁₀arylR^{d}, C₁₋₆alkylNHC(O)C₆₋₁₀arylR^{d}, C₁₋₆alkylOC₁₋₆alkylC₅₋₆arylR^{d}, C₁₋₆alkylSC₁₋₆alkylC₅₋₆arylR^{d}, C₁₋₆alkylC₅₋₉heterocyclylR^{d}, C₁₋₆alkylC₃₋₉cycloalkylR^{d}, C₁₋₆alkylNHC₁₋₆alkylC₅₋₉heterocyclylR^{d}, C₁₋₆alkylNHC₅₋₉heterocyclyl(R^{d})ₜ, C₁₋₆alkylNHC(O)C₅₋₉heterocyclylR^{d}, C₁₋₆alkylOC₁₋₆alkylC₅₋₉heterocyclylR^{d}, C₁₋₆alkylSC₁₋₆alkylC₅₋₉heterocyclylR^{d}, C₁₋₆alkylNHC₁₋₆alkylC₃₋₉cycloalkylR^{d}, C₁₋₆alkylOC₁₋₆alkylC₃₋₉cycloalkylR^{d}, or C₁₋₆alkylSC₁₋₆alkylC₃₋₉cycloalkylR^{d};
each R⁴ is, independently, selected from H, halogen, C₁₋₆alkyl, C₃₋₁₂cycloalkyl, C₆₋₁₀aryl, C₁₋₆alkyl-C₆₋₁₀aryl, C₅₋₁₀heterocyclyl, or C₁₋₆alkyl-C₅₋₁₀heterocyclyl wherein the C₁₋₆alkyl, C₃₋₁₂cycloalkyl, C₆₋₁₀aryl, C₁₋₆alkyl-C₆₋₁₀aryl, C₅₋₁₀heterocyclyl, and C₁₋₆alkyl-C₅₋₁₀heterocyclyl is optionally substituted by 1, 2, or 3 substitutents independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂S(═O)C₁₋₆alkyl-R^{a}, S(═O)NHC₁₋₆alkyl-R^{a}, S(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-R^{a}, N(C₁₋₆alkyl-R³)₂, or NHC(═O)C₁₋₆alkyl;
t is 0, 1, 2, 3, 4 or 5;
each R^{c} is, independently, selected from H, C(═O)C₁₋₄alkyl, C(═O)C₁₋₄alkylOC₁₋₄alkyl, C(═O)C₁₋₄alkylC(═O)OC₁₋₄alkyl, C(═O)C₁₋₄alkylC(═O)OH, C(═O)C₁₋₄alkylOC(═O)C₁₋₄alkyl, C₅₋₆arylR^{d}, C₅₋₉heterocyclylR^{d}, C₃₋₉cycloalkylR^{d}, C(═O)C₅₋₆arylR^{d}, C(═O)C₅₋₉heterocyclylR^{d}, C(═O)C₃₋₉cycloalkylR^{d}, C₁₋₄alkyl-C₅₋₆arylR^{d}, C₁₋₄alkyl-C₆₋₉heterocyclylR^{d}, or C₁₋₄alkyl-C₃₋₉cycloalkylR^{d}; and
R^{d} is selected from H, C₁₋₃alkyl, NH₂, OH, COOH, OC₁₋₃alkyl, or OC₁₋₃alkylOH; provided that when the compound has formula Ib, W is N, u is 1, and R³ is H, then [R¹-(V)ₙ]ₘ-Q is other than polyC₁₋₄alkyl substituted cyclohexenyl or a nitro substituted furyl.

2. A compound of claim 1 wherein the compound has the structure of the formula Ia.

3. A compound of claim 1 wherein the compound has the structure of the formula Ib.

4. A compound of claim 1 wherein W is N.

5. A compound of claim 1 wherein R³ is selected from H, C₁₋₆alkyl, C₁₋₆alkylNR^{c}R^{c}, C₁₋₆alkylOR^{c}, C₁₋₆alkylNHC₁₋₆alkylC₆₋₁₀arylR^{d}, C₁₋₆alkylNHC(O)C₆₋₁₀arylR^{d}, C₁₋₆alkylOC₁₋₆alkylC₅₋₆arylR^{d}, C₁₋₆alkylC₆₋₁₀arylR^{d}, C₁₋₆alkylC₅₋₉heterocyclylR^{d}, or C₁₋₆alkylC₃₋₉cycloalkylR^{d}.

6. A compound of claim 1 wherein R³ is selected from H, C₁₋₆alkyl, C₁₋₆alkylNR^{c}R^{c}, or C₁₋₆alkyl-C₅₋₉heterocyclylR^{d}.

7. A compound of claim 1 wherein R³ is C₁₋₃alkyl.

8. A compound of claim 1 wherein Q is C₆₋₁₀aryl, C₃₋₁₀cycloalkyl or C₃₋₁₀cycloalkenyl.

9. A compound of claim 1 wherein Q is C₆aryl or C₃₋₁₀cycloalkenyl.

10. A compound of claim 1 wherein -[X]_{q}-[Y]ᵣ- is OC₁₋₃alkyl, N(C₁₋₃alkyl)C₁₋₃alkyl, C₁₋₃alkylOC₁₋₃alkyl, C₁₋₃alkylN(H)C₁₋₃alkyl or C₁₋₃alkyl optionally substituted by OH.

11. A compound of claim 1 wherein q is 0, r is 0, and each R⁴ is H.

12. A compound of claim 1 wherein m is 1, V is S, n is 0 or 1, and R¹ is C₆₋₁₀aryl or C₅₋₁₅heterocyclyl, wherein each the aryl and heterocyclyl is optionally substituted by 1 or 2 substituents independently selected from: halogen, CN, C₁₋₄alkyl, C₁₋₄haloalkyl, OC₁₋₄alkyl, OC₁₋₄haloalkyl, —C(O)H, COOH, OC₁₋₄alkyl-C₆₋₁₀aryl, OH, NHC(═O)C₁₋₄alkyl and —C₆aryl-OC₁₋₄alkyl.

13. A compound of claim 1 wherein m is 1, n is 0, and R¹ is C₆₋₁₀aryl, wherein the aryl is optionally substituted by 1 or 2 substituents independently selected from: halogen, CN, C₁₋₄alkyl, C₁₋₄haloalkyl, OC₁₋₄alkyl, OC₁₋₄haloalkyl, —C(O)H, COOH, OC₁₋₄alkyl-C₆₋₁₀aryl, OH, NHC(═O)C₁₋₄alkyl and —C₆aryl-OC₁₋₄alkyl.

14. A compound of claim 1 wherein:
R¹ is, independently, selected from H, halogen, C₆aryl, or C₅₋₆heterocyclyl wherein the C₆aryl, or C₅₋₆heterocyclyl is optionally substituted by 1, 2, or 3 substituents, independently, selected from: halogen, OH, NH₂, CN, C(═O)NH₂, C₁₋₆alkyl, OC₁₋₆alkyl, C₁₋₄alkylOH, C₁₋₄alkylOC₁₋₃alkyl, CH₂OH, SO₂H, SO₂NHC(CH₃)₃, SO₂C₁₋₆alkyl, SO₂NHC₁₋₆alkyl, OC₁₋₃alkylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₅₋₆heterocyclyl, OC₅₋₆aryl, —C₆aryl-OC₁₋₄alkyl or OC₁₋₆alkyl-C₅₋₆aryl; and
R² is H or C₁₋₆alkyl;
R³ is H or C₁₋₃alkyl; and
each R⁴ is H.

15. A compound of claim 1 wherein:
Q is C₆aryl or C₅₋₉heterocyclyl;
W is N;
R¹ is, independently, selected from H, halogen, C₆aryl, or C₅₋₆heterocyclyl wherein the C₆aryl, or C₅₋₆heterocyclyl is optionally substituted by 1, 2, or 3 substituents, independently, selected from: halogen, OH, NH₂, CN, C(═O)NH₂, C₁₋₆alkyl, OC₁₋₆alkyl, C₁₋₄alkylOH, C₁₋₄alkylOC₁₋₃alkyl, CH₂OH, SO₂H, SO₂NHC(CH₃)₃, SO₂C₁₋₆alkyl, SO₂NHC₁₋₆alkyl, OC₁₋₃alkylOC₁₋₃alkyl, OC₁₋₃alkylOH, OC₁₋₃alkylOC(═O)C₁₋₃alkyl, C(═O)C₁₋₆alkyl, C(═O)OC₁₋₆alkyl, C(═O)NH₂, C₅₋₆heterocyclyl, OC₅₋₆aryl, —C₆aryl-OC₁₋₄alkyl or OC₁₋₆alkyl-C₅₋₆aryl; and
R² is C₁₋₃alkyl.

16. A compound of claim 3 wherein:
Q is C₆₋₁₀aryl;
W is N;
-[X]_{q}-[Y]ᵣ- is OC₁₋₃alkyl;
m is 1;
n is 0; and
R¹ is C₆₋₁₀aryl optionally substituted by 1 or 2 substituents independently selected from: OC₁₋₄alkyl and —C₆aryl-OC₁₋₄alkyl.

17. A compound of claim 3 wherein:
Q is C₃₋₁₀cycloalkenyl;
W is N
-[X]_{q}-[Y]ᵣ- is absent;
m is 1;
n is 0; and
R¹ is C₆₋₁₀aryl optionally substituted by 1 or 2 substituents independently selected from: OC₁₋₄alkyl and —C₁₋₆aryl-OC₁₋₄alkyl.

18. A compound of claim 2 wherein:
Q is C₆₋₁₀aryl, C₃₋₁₀cycloalkyl or C₃₋₁₀cycloalkenyl;
W is N;
-[X]_{q}-[Y]ᵣ- is OC₁₋₃alkyl, N(C₁₋₃alkyl)C₁₋₃alkyl, C₁₋₃alkylOC₁₋₃alkyl, C₁₋₃alkylN(H)C₁₋₃alkyl or C₁₋₃alkyl optionally substituted by OH;
m is 1;
V is S;
n is 0 or 1; and
R¹ is C₆₋₁₀aryl or C₅₋₁₅heterocyclyl, wherein each the aryl and heterocyclyl is optionally substituted by 1 or 2 substituents independently selected from: halogen, CN, C₁₋₄alkyl, C₁₋₄haloalkyl, OC₁₋₄alkyl, OC₁₋₄haloalkyl, —C(O)H, COOH, OC₁₋₄alkyl-C₆₋₁₀aryl, OH, NHC(═O)C₁₋₄alkyl and —C₆aryl-OC₁₋₄alkyl.

19. A compound selected from:
2-Amino-6-[(Z)-2-(3′-methoxybiphenyl-3-yl)vinyl]-3-methylpyrimidin-4(3H)-one;
2-Amino-6-[(E)-2-(3′-methoxybiphenyl-3-yl)vinyl]-3-methylpyrimidin-4(3H)-one;
2-Amino-6-[(Z)-2-(3-bromophenyl)vinyl]-3-methylpyrimidin-4(3H)-one and
2-Amino-6-[(E)-2-(3-bromophenyl)vinyl]-3-methylpyrimidin-4(3H)-one;
or a pharmaceutically acceptable salt, tautomer, or in vivo-hydrolysable precursor thereof.

20. A pharmaceutical composition comprising as active ingredient a therapeutically effective amount of a compound according to claim 1 or 19 in association with a pharmaceutically acceptable excipient, carrier or diluent.

21-25. (canceled)

26. A method of inhibiting activity of BACE comprising contacting said BACE with a compound of claim 1 or 19.

27. A method of treating or preventing an Aβ-related pathology in a mammal, comprising administering to said patient a therapeutically effective amount of a compound of claim 1 or 19.

28. The method of claim 27, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

29. The method of claim 27, wherein said mammal is a human.

30. A method of treating or preventing an Aβ-related pathology in a mammal, comprising administering to said patient a therapeutically effective amount of a compound of claim 1 or 19 and at least one cognitive enhancing agent, memory enhancing agent, or choline esterase inhibitor.

31. The method of claim 30, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

32. The method of claim 30, wherein said mammal is a human.
